SEQUENCE LISTING (6 sequences)

SEQ ID NO: 1 - DNA, 7149 bp, Homo sapiens
atgtctggcggcgccgcagagaagcagagcagcactcccggttccctgttcctctcgccgccggctcctgcccccaagaatggctccagctccgattcctccgtgggggagaaactgggagccgcggccgccgacgctgtgaccggcaggaccgaggagtacaggcgccgccgccacactatggacaaggacagccgtggggcggccgcgaccactaccaccactgagcaccgcttcttccgccggagcgtcatctgcgactccaatgccactgcactggagcttcccggccttcctctttccctgccccagcccagcatccccgcggctgtcccgcagagtgctccaccggagccccaccgggaagagaccgtgaccgccaccgccacttcccaggtagcccagcagcctccagccgctgccgcccctggggaacaggccgtcgcgggccctgccccctcgactgtccccagcagtaccagcaaagaccgcccagtgtcccagcctagccttgtggggagcaaagaggagccgccgccggcgagaagtggcagcggcggcggcagcgccaaggagccacaggaggaacggagccagcagcaggatgatatcgaagagctggagaccaaggccgtgggaatgtctaacgatggccgctttctcaagtttgacatcgaaatcggcagaggctcctttaagacggtctacaaaggtctggacactgaaaccaccgtggaagtcgcctggtgtgaactgcaggatcgaaaattaacaaagtctgagaggcagagatttaaagaagaagctgaaatgttaaaaggtcttcagcatcccaatattgttagattttatgattcctgggaatccacagtaaaaggaaagaagtgcattgttttggtgactgaacttatgacgtctggaacacttaaaacgtatctgaaaaggtttaaagtgatgaagatcaaagttctaagaagctggtgccgtcagatccttaaaggtcttcagtttcttcatactcgaactccacctatcattcaccgcgatcttaaatgtgacaacatctttatcaccggccctactggctcagtcaagattggagacctcggtctggcaaccctgaagcgggcttcttttgccaagagtgtgataggtaccccagagttcatggcccctgagatgtatgaggagaaatatgatgaatccgttgacgtttatgcttttgggatgtgcatgcttgagatggctacatctgaatatccttactcggagtgccaaaatgctgcgcagatctaccgtcgcgtgaccagtggggtgaagccagccagttttgacaaagtagcaattcctgaagtgaaggaaattattgaaggatgcatacgacaaaacaaagatgaaagatattccatcaaagaccttttgaaccatgccttcttccaagaggaaacaggagtacgggtagaattagcagaagaagatgatggagaaaaaatagccataaaattatggctacgtattgaagatattaagaaattaaagggaaaatacaaagataatgaagctattgagttttcttttgatttagagagagatgtcccagaagatgttgcacaagaaatggtagagtctgggtatgtctgtgaaggtgatcacaagaccatggctaaagctatcaaagacagagtatcattaattaagaggaaacgagagcagcggcagttggtacgggaggagcaagaaaaaaaaaagcaggaagagagcagtctcaaacagcaggtagaacaatccagtgcttcccagacaggaatcaagcagctcccttctgctagcaccggcatacctactgcttctaccacttcagcttcagtttctacacaagtagaacctgaagaacctgaggcagatcaacatcaacaactacagtaccagcaacccagtatatctgtgttatctgatgggacggttgacagtggtcagggatcctctgtcttcacagaatctcgagtgagcagccaacagacagtttcatatggttcccaacatgaacaggcacattctacaggcacagtcccagggcatataccttctactgtccaagcacagtctcagccccatggggtatatccaccctcaagtgtggcacaggggcagagccagggtcagccatcctcaagtagcttaacaggggtttcatcttcccaacccatacaacatcctcagcagcagcagggaatacagcagacagcccctcctcaacagacagtgcagtattcactttcacagacatcaacctccagtgaggccactactgcacagccagtgagtcagcctcaagctccacaagtcttgcctcaagtatcagctggaaaacagcttccagtttcccagccagtaccaactatccaaggcgaacctcagatcccagttgcgacacaaccctcggttgttccagtccactctggtgctcatttccttccagtgggacagccgctccctactcccttgctccctcagtaccctgtctctcagattcccatatcaactcctcatgtgtctacggctcagacaggtttctcatcccttcccatcacaatggcagctggcattactcagcctctgctcacgttggcttcatctgctacaacagctgcgatcccgggggtatcaactgtggttcctagtcagcttccaacccttctgcagcctgtgactcagctgccaagtcaggttcacccacagctcctacaaccagcagttcagtccatgggaataccagctaaccttggacaagctgctgaggttccactttcctctggagatgttctgtaccagggcttcccacctcgactgccaccacagtacccaggagattcaaatattgctccctcttccaacgtggcttctgtttgcatccattctacagtcctatcccctcccatgccgacagaagtactggctacacctgggtactttcccacagtggtgcagccttatgtggaatcaaatcttttagttcctatgggtggtgtaggaggacaggttcaagtgtcccagccaggagggagtttagcacaagcccccactacatcctcccagcaagcagttttggagagtactcagggagtctctcaggttgctcctgcagagccagttgcagtagcacagccccaagctacccagccgaccactttggcttcctctgtagacagtgcacattcagatgttgcttcaggtatgagtgatggcaatgagaacgtcccatcttccagtggaaggcatgaaggaagaactacaaaacggcattaccgaaaatctgtaaggagtcgctctcgacatgaaaaaacttcacgcccaaaattaagaattttgaatgtttcaaataaaggagaccgagtagtagaatgtcaattagagactcataataggaaaatggttacattcaaatttgacctagatggtgacaaccccgaggagatagcaacaattatggtgaacaatgactttattctagcaatagagagagagtcgtttgtggatcaagtgcgagaaattattgaaaaagctgatgaaatgctcagtgaggatgtcagtgtggaaccagagggtgatcagggattggagagtctacaaggaaaggatgactatggcttttcaggttctcagaaattggaaggagagttcaaacaaccaattcctgcgtcttccatgccacagcaaataggcattcctaccagttctttaactcaagttgttcattctgcgggaaggcggtttatagtgagtcctgtgccagaaagccgattacgagaatcaaaagttttccccagtgaaataacagatacagttgctgcctctacagctcagagccctggaatgaacttgtctcactctgcatcatcccttagtctacaacaggccttttctgaacttagacgtgcccaaatgacagaaggacccaatacagcacctccaaactttagtcatacaggaccaacatttccagtagtacctcctttcttaagtagcattgctggagtcccaaccacagcagcagccacagcaccagtccctgcaacaagcagccctcctaatgacatttccacatcagtaattcagtctgaggttacagtgcccactgaagaggggattgctggagttgccaccagcacaggtgtggtaacttcaggtggtctccccataccacctgtgtctgaatcaccagtactttccagcgtagtttcaagtatcacaatacctgcagttgtctcaatatctactacatccccgtcacttcaagtccccacatccacatctgagatcgttgtttctagtacagcactgtatccttcagtaacagtttcagcaacttcagcctctgcagggggcagtactgctaccccaggtcctaagcctccagctgtagtatctcagcaggcagcaggcagcactactgtgggagccacattaacatcagtttctaccaccacttcattcccaagcacagcttcacagctgtccattcagcttagcagcagtacttctactcctactttagctgaaaccgtggtagttagcgcacactcactagataagacatctcatagcagtacaactggattggctttctccctctctgcaccatcttcctcttcctctcctggagcaggagtgtctagttatatttctcagcctggtgggctgcatcctttggtcattccatcagtgatagcttctactcctattcttccccaagcagcaggacctacttctacacctttattaccccaagtacctagtatcccacccttggtacagcctgttgccaatgtgcctgctgtacagcagacactaattcatagtcagcctcaaccagctttgcttcccaaccagccccatactcattgtcctgaagtagattctgatacacaacccaaagctcctggaattgatgacataaagactctagaagaaaagctgcggtctctgttcagtgaacacagctcatctggagctcagcatgcctctgtctcactggagacctcactagtcatagagagcactgtcacaccaggcatcccaactactgctgttgcaccaagcaaactcctgacttctaccacaagtacttgcttaccaccaaccaatttaccactaggaacagttgctttgccagttacaccagtggtcacacctgggcaagtttctaccccagtcagcactactacatcaggagtgaaacctggaactgctccctccaagccacctctaactaaggctccggtgctgccagtgggtactgaacttccagcaggtactctacccagcgagcagctgccaccttttccaggaccttctctaacccagtcccagcaacctctagaggatcttgatgctcaattgagaagaacacttagtccagagatgatcacagtgacttctgcggttggtcctgtgtccatggcggctccaacagcaatcacagaagcaggaacacagcctcagaagggtgtttctcaagtcaaagaaggccctgtcctagcaactagttcaggagctggtgtttttaagatgggacgatttcaggtttctgttgcagcagacggtgcccagaaagagggtaaaaataagtcagaagatgcaaagtctgttcattttgaatccagcacctcagagtcctcagtgctatcaagtagtagtccagagagtaccttggtgaaaccagagccgaatggcataaccatccctggtatctcttcagatgtgccagagagtgcccacaaaactactgcctcagaggcaaagtcagacactgggcagcctaccaaggttggacgttttcaggtgacaactacagcaaacaaagtgggtcgtttctctgtatcaaaaactgaggacaagatcactgacacaaagaaagaaggaccagtggcatctcctccttttatggatttggaacaagctgttcttcctgctgtgataccaaagaaagagaagcctgaactgtcagagccttcacatctaaatgggccgtcttctgacccggaggccgcttttttaagtagggatgtggatgatggttccggtagtccacactcgccccatcagctgagctcaaagagccttcctagccagaatctaagtcaaagccttagtaattcatttaactcctcttacatgagtagcgacaatgagtcagatatcgaagatgaagacttaaagttagagctgcgacgactacgagataaacatctcaaagagattcaggacctgcagagtcgccagaagcatgaaattgaatctttgtataccaaactgggcaaggtgccccctgctgttattattcccccagctgctcccctttcagggagaagacgacgacccactaaaagcaaaggcagcaaatctagtcgaagcagttccttggggaataaaagcccccagctttcaggtaacctgtctggtcagagtgcagcttcagtcttgcacccccagcagaccctccaccctcctggcaacatcccagagtccgggcagaatcagctgttacagccccttaagccatctccctccagtgacaacctctattcagccttcaccagtgatggtgccatttcagtaccaagcctttctgctccaggtcaaggaaccagcagcacaaacactgttggggcaacagtgaacagccaagccgcccaagctcagcctcctgccatgacgtccagcaggaagggcacattcacagatgacttgcacaagttggtagacaattgggcccgagatgccatgaatctctcaggcaggagaggaagcaaagggcacatgaattacgagggccctggaatggcaaggaagttctctgcacctgggcaactgtgcatctccatgacctcgaacctgggtggctctgcccccatctctgcagcatcagctacctctctaggtcacttcaccaagtctatgtgccccccacagcagtatggctttccagctaccccatttggcgctcaatggagtgggacgggtggcccagcaccacagccacttggccagttccaacctgtgggaactgcctccttgcagaatttcaacatcagcaatttgcagaaatccatcagcaaccccccaggctccaacctgcggaccacttag

SEQ ID NO: 2 - protein, 2382 aa, Homo sapiens
MSGGAAEKQSSTPGSLFLSPPAPAPKNGSSSDSSVGEKLGAAAADAVTGRTEEYRRRRHTMDKDSRGAAATTTTTEHRFFRRSVICDSNATALELPGLPLSLPQPSIPAAVPQSAPPEPHREETVTATATSQVAQQPPAAAAPGEQAVAGPAPSTVPSSTSKDRPVSQPSLVGSKEEPPPARSGSGGGSAKEPQEERSQQQDDIEELETKAVGMSNDGRFLKFDIEIGRGSFKTVYKGLDTETTVEVAWCELQDRKLTKSERQRFKEEAEMLKGLQHPNIVRFYDSWESTVKGKKCIVLVTELMTSGTLKTYLKRFKVMKIKVLRSWCRQILKGLQFLHTRTPPIIHRDLKCDNIFITGPTGSVKIGDLGLATLKRASFAKSVIGTPEFMAPEMYEEKYDESVDVYAFGMCMLEMATSEYPYSECQNAAQIYRRVTSGVKPASFDKVAIPEVKEIIEGCIRQNKDERYSIKDLLNHAFFQEETGVRVELAEEDDGEKIAIKLWLRIEDIKKLKGKYKDNEAIEFSFDLERDVPEDVAQEMVESGYVCEGDHKTMAKAIKDRVSLIKRKREQRQLVREEQEKKKQEESSLKQQVEQSSASQTGIKQLPSASTGIPTASTTSASVSTQVEPEEPEADQHQQLQYQQPSISVLSDGTVDSGQGSSVFTESRVSSQQTVSYGSQHEQAHSTGTVPGHIPSTVQAQSQPHGVYPPSSVAQGQSQGQPSSSSLTGVSSSQPIQHPQQQQGIQQTAPPQQTVQYSLSQTSTSSEATTAQPVSQPQAPQVLPQVSAGKQLPVSQPVPTIQGEPQIPVATQPSVVPVHSGAHFLPVGQPLPTPLLPQYPVSQIPISTPHVSTAQTGFSSLPITMAAGITQPLLTLASSATTAAIPGVSTVVPSQLPTLLQPVTQLPSQVHPQLLQPAVQSMGIPANLGQAAEVPLSSGDVLYQGFPPRLPPQYPGDSNIAPSSNVASVCIHSTVLSPPMPTEVLATPGYFPTVVQPYVESNLLVPMGGVGGQVQVSQPGGSLAQAPTTSSQQAVLESTQGVSQVAPAEPVAVAQPQATQPTTLASSVDSAHSDVASGMSDGNENVPSSSGRHEGRTTKRHYRKSVRSRSRHEKTSRPKLRILNVSNKGDRVVECQLETHNRKMVTFKFDLDGDNPEEIATIMVNNDFILAIERESFVDQVREIIEKADEMLSEDVSVEPEGDQGLESLQGKDDYGFSGSQKLEGEFKQPIPASSMPQQIGIPTSSLTQVVHSAGRRFIVSPVPESRLRESKVFPSEITDTVAASTAQSPGMNLSHSASSLSLQQAFSELRRAQMTEGPNTAPPNFSHTGPTFPVVPPFLSSIAGVPTTAAATAPVPATSSPPNDISTSVIQSEVTVPTEEGIAGVATSTGVVTSGGLPIPPVSESPVLSSVVSSITIPAVVSISTTSPSLQVPTSTSEIVVSSTALYPSVTVSATSASAGGSTATPGPKPPAVVSQQAAGSTTVGATLTSVSTTTSFPSTASQLSIQLSSSTSTPTLAETVVVSAHSLDKTSHSSTTGLAFSLSAPSSSSSPGAGVSSYISQPGGLHPLVIPSVIASTPILPQAAGPTSTPLLPQVPSIPPLVQPVANVPAVQQTLIHSQPQPALLPNQPHTHCPEVDSDTQPKAPGIDDIKTLEEKLRSLFSEHSSSGAQHASVSLETSLVIESTVTPGIPTTAVAPSKLLTSTTSTCLPPTNLPLGTVALPVTPVVTPGQVSTPVSTTTSGVKPGTAPSKPPLTKAPVLPVGTELPAGTLPSEQLPPFPGPSLTQSQQPLEDLDAQLRRTLSPEMITVTSAVGPVSMAAPTAITEAGTQPQKGVSQVKEGPVLATSSGAGVFKMGRFQVSVAADGAQKEGKNKSEDAKSVHFESSTSESSVLSSSSPESTLVKPEPNGITIPGISSDVPESAHKTTASEAKSDTGQPTKVGRFQVTTTANKVGRFSVSKTEDKITDTKKEGPVASPPFMDLEQAVLPAVIPKKEKPELSEPSHLNGPSSDPEAAFLSRDVDDGSGSPHSPHQLSSKSLPSQNLSQSLSNSFNSSYMSSDNESDIEDEDLKLELRRLRDKHLKEIQDLQSRQKHEIESLYTKLGKVPPAVIIPPAAPLSGRRRRPTKSKGSKSSRSSSLGNKSPQLSGNLSGQSAASVLHPQQTLHPPGNIPESGQNQLLQPLKPSPSSDNLYSAFTSDGAISVPSLSAPGQGTSSTNTVGATVNSQAAQAQPPAMTSSRKGTFTDDLHKLVDNWARDAMNLSGRRGSKGHMNYEGPGMARKFSAPGQLCISMTSNLGGSAPISAASATSLGHFTKSMCPPQQYGFPATPFGAQWSGTGGPAPQPLGQFQPVGTASLQNFNISNLQKSISNPPGSNLRTT

SEQ ID NO: 3 - DNA, 3732 bp, Homo sapiens
atgttggcatccccggccacggagaccaccgtcctcatgtcccagactgaggccgacctggccctgcggcccccgcctcctcttggcaccgcggggcagccccgcctcgggccccctcctcgccgagcgcgccgcttctccgggaaggctgagccccggccgcgctcttctcgtctcagccgccgtagctcagtcgacttggggctgctgagctcttggtccctgccagcctcacccgctccggacccccccgatcctccggactccgctggtcctggccccgcgaggagcccaccgcctagctccaaagaaccccccgagggcacgtggaccgagggagcccctgtgaaggctgcggaagactccgcgcgtcccgagctcccggactctgcagtgggcccggggtccagggagccgctaagggtccctgaagctgtggccctagagcggcggcgggagcaggaagaaaaggaggacatggagacccaggctgtggcaacgtcccccgatggccgatacctcaagtttgacatcgagattggacgtggctccttcaagacggtgtatcgagggctagacaccgacaccacagtggaggtggcctggtgtgagctgcagactcggaaactgtctagagctgagcggcagcgcttctcagaggaggtggagatgctcaaggggctgcagcaccccaacatcgtccgcttctatgattcgtggaagtcggtgctgaggggccaggtttgcatcgtgctggtcaccgaactcatgacctcgggcacgctcaagacgtacctgaggcggttccgggagatgaagccgcgggtccttcagcgctggagccgccaaatcctgcggggacttcatttcctacactcccgggttcctcccatcctgcaccgggatctcaagtgcgacaatgtctttatcacgggacctactggctctgtcaaaatcggggacctgggcctggccacgctcaagcgcgcctcctttgccaagagtgtcatcgggaccccggaattcatggcccccgagatgtacgaggaaaagtacgatgaggccgtggacgtgtacgcgttcggcatgtgcatgctggagatggccacctctgagtacccgtactccgagtgccagaatgccgcgcaaatctaccgcaaggtcacttcgggcagaaagccgaacagcttccacaaggtgaagatacccgaggtgaaggagatcattgaaggctgcatccgcacggataagaacgagaggttcaccatccaggacctcctggcccacgccttcttccgcgaggagcgcggtgtgcacgtggaactagcggaggaggacgacggcgagaagccgggcctcaagctctggctgcgcatggaggacgcgcggcgcggggggcgcccacgggacaaccaggccatcgagttcctgttccagctgggccgggacgcggccgaggaggtggcacaggagatggtggctctgggcttggtctgtgaagccgattaccagccagtggcccgtgcagtacgtgaacgggttgctgccatccagcgaaagcgtgagaagctgcgtaaagcaagggaattggaggcactcccaccagagccaggacctccaccagcaactgtgcccatggcccccggtccccccagtgtcttcccccctgagcctgaggagccagaggcagaccagcaccagcccttccttttccgccacgccagctactcatctaccacttcggattgcgagactgatggctacctcagctcctccggcttcctggatgcctcagaccctgcccttcagccccctgggggggtgccatccagcctggctgagtcccatctctgcctgccctcggcttttgccctatccattccacgttctggccctggaagtgacttttcccccggggacagctatgcctcagatgcagcttcaggccttagcgatgtgggagaagggatgggacaaatgaggagacccccagggaggaatctccggcgcagaccccgatcccggctgcgggtcactagtgtctcagaccagaatgacagagtggttgagtgccagctacagacccataacagcaagatggtgaccttccgatttgatctggatggggacagcccggaagagattgcagctgccatggtatataacgagttcattctgccttcggagcgagatggatttctcagacggattcgggagattatccagcgagtggagaccctgttgaagagagacactggccccatggaggctgctgaagacaccctaagcccccaggaggagccagcaccattacctgccctgcccgtccccctcccagacccatccaatgaagagctccagagcagcacctccctggagcacaggagctggacagccttctccacctcctcatcttctcctggaactcctttgtctcctggaaacccattttcccctggaacccccatttccccaggtcccatcttccccatcacttctcccccatgtcatcccagcccctccccattctcccccatttcttcccaggtctcctcaaatccctctccacaccccaccagctctccacttccattctcctccagcacacccgagtttccggtcccactctctcagtgtccctggagttctctccccacgacttctccacctacgttctctcccacttgttctcaggtcactcttagttcccctttctttcctccgtgcccctccacttcttccttcccctccaccacagcagcccctctcctttctctggctagtgccttctcactggctgtgatgactgtggcccagtccctgctgtccccctcacctgggctcctttcccagtctcctccagcccctcctagtcccctccctagcctgccccttccccctcccgttgctcctggtggccaggaaagcccttcaccccacacagctgaggtggagagtgaggcctcaccacctcctgctcggcccctcccaggggaagccaggctggcgcccatctctgaagagggaaagccgcagcttgttgggcgtttccaagtgacttcatccaaggaaccggctgagcctcttcccttgcagccaacatcccccactctctctggttctccaaaaccttcaacccctcagctcacttcagagagctcagatacagaggacagtgctggaggcgggccagagaccagggaagctctggctgagagcgaccgtgcagctgagggtctgggggctggagttgaggaggaaggagatgatgggaaggaaccccaagttgggggcagcccccaacccctgagccatcccagcccagtgtggatgaactactcctacagcagcctgtgtttgagcagcgaggagtcagaaagcagtggggaagatgaggagttctgggctgagctgcagagtcttcggcagaagcacttgtcagaggtggaaacactacagacactacagaaaaaagaaattgaagatttgtacagccggctggggaagcagcccccaccgggtattgtggccccagctgctatgctgtccagccgccagcgccgcctctccaagggcagcttccccacctcccgccgcaacagcctacagcgctctgagcccccaggccctggcatcatgcgaaggaactctctgagtggcagcagcaccggctcccaggagcagcgggcaagcaagggggtgacattcgccggggatgttggcaggatgtga

SEQ ID NO: 4 - protein, 1243 aa, Homo sapiens
MLASPATETTVLMSQTEADLALRPPPPLGTAGQPRLGPPPRRARRFSGKAEPRPRSSRLSRRSSVDLGLLSSWSLPASPAPDPPDPPDSAGPGPARSPPPSSKEPPEGTWTEGAPVKAAEDSARPELPDSAVGPGSREPLRVPEAVALERRREQEEKEDMETQAVATSPDGRYLKFDIEIGRGSFKTVYRGLDTDTTVEVAWCELQTRKLSRAERQRFSEEVEMLKGLQHPNIVRFYDSWKSVLRGQVCIVLVTELMTSGTLKTYLRRFREMKPRVLQRWSRQILRGLHFLHSRVPPILHRDLKCDNVFITGPTGSVKIGDLGLATLKRASFAKSVIGTPEFMAPEMYEEKYDEAVDVYAFGMCMLEMATSEYPYSECQNAAQIYRKVTSGRKPNSFHKVKIPEVKEIIEGCIRTDKNERFTIQDLLAHAFFREERGVHVELAEEDDGEKPGLKLWLRMEDARRGGRPRDNQAIEFLFQLGRDAAEEVAQEMVALGLVCEADYQPVARAVRERVAAIQRKREKLRKARELEALPPEPGPPPATVPMAPGPPSVFPPEPEEPEADQHQPFLFRHASYSSTTSDCETDGYLSSSGFLDASDPALQPPGGVPSSLAESHLCLPSAFALSIPRSGPGSDFSPGDSYASDAASGLSDVGEGMGQMRRPPGRNLRRRPRSRLRVTSVSDQNDRVVECQLQTHNSKMVTFRFDLDGDSPEEIAAAMVYNEFILPSERDGFLRRIREIIQRVETLLKRDTGPMEAAEDTLSPQEEPAPLPALPVPLPDPSNEELQSSTSLEHRSWTAFSTSSSSPGTPLSPGNPFSPGTPISPGPIFPITSPPCHPSPSPFSPISSQVSSNPSPHPTSSPLPFSSSTPEFPVPLSQCPWSSLPTTSPPTFSPTCSQVTLSSPFFPPCPSTSSFPSTTAAPLLSLASAFSLAVMTVAQSLLSPSPGLLSQSPPAPPSPLPSLPLPPPVAPGGQESPSPHTAEVESEASPPPARPLPGEARLAPISEEGKPQLVGRFQVTSSKEPAEPLPLQPTSPTLSGSPKPSTPQLTSESSDTEDSAGGGPETREALAESDRAAEGLGAGVEEEGDDGKEPQVGGSPQPLSHPSPVWMNYSYSSLCLSSEESESSGEDEEFWAELQSLRQKHLSEVETLQTLQKKEIEDLYSRLGKQPPPGIVAPAAMLSSRQRRLSKGSFPTSRRNSLQRSEPPGPGIMRRNSLSGSSTGSQEQRASKGVTFAGDVGRM

SEQ ID NO: 5 - protein, 17 aa, Artificial Sequence
  WNK1 Immunizing Peptide
SQPGGSLAQAPTTSSQQ

SEQ ID NO: 6 - protein, 14 aa, Artificial Sequence
  WNK4 Immunizing Peptide
MGQMRRPPGRNLRR

(19) United States
(12) Patent Application Publication
(10) Pub. No.: US 2003/0082720 A1
(43) Pub. Date: May 1, 2003

(54) Compositions methods and kits relating to treating and diagnosing hypertension

(76) Inventors: Richard P. Lifton, Guilford, CT (US); Frederick H. Wilson, New Haven, CT (US); Keith Choate, New Haven, CT (US); Carol Nelson-Williams, Hamden, CT (US); Kazuhiko Ishikawa, Toyonaka City (JP)

Correspondence Address: MORGAN, LEWIS & BOCKIUS LLP, 1701 MARKET STREET, PHILADELPHIA, PA, 19103-2921, US

(21) Appl. No.: 10/196,935

(22) Filed: Jul. 17, 2002

Related U.S. Application Data
(60) Provisional application No. 60/306,084, filed on Jul. 17, 2001.

(51) Int. Cl. C12N 9/12; C07H 21/04; C12P 21/02; C12N 5/06

(52) U.S. Cl. 435/69.1; 435/194; 435/320.1; 435/325; 536/23.2

(57) ABSTRACT

The present invention relates to novel nucleic acids encoding a mammalian WNK, and proteins encoded thereby, preferably, human WNK1 and human WNK4. These novel nucleic acids, and mutant forms thereof, are associated with, inter alia, renal electrolyte handling, hypertension, and pseudohypoaldosterism type II (PHA II). That is, the present invention relates to novel mutations (e.g., deletions and missense mutations in an exon, intron, or both, of a nucleic acid encoding a WNK) that mediate and/or are associated with altered expression, among other things. These mutations are, in turn, associated with and/or mediate disease (e.g., hypertension, PHA II, and the like). Thus, these nucleic acids provide a novel target for treatment, diagnosis, and development of therapeutics to treat these diseases.

### CROSS-REFERENCE TO RELATED APPLICATIONS

This application is entitled to priority pursuant to 35 U.S.C. §119(e) to U.S. Provisional Patent Application No. 60/306,084, which was filed on Jul. 17, 2001.

### STATEMENT REGARDING FEDERALLY SUPPORTED RESEARCH OR DEVELOPMENT

This research was supported in part by U.S. Government funds (NIH Grant No. 2P50 HL55007), and the U.S. Government may therefore have certain rights in the invention.

### BACKGROUND OF THE INVENTION

Hypertension, or high blood pressure, is often referred to as the silent killer in that a hypertensive patient often exhibits no specific symptoms, yet hypertension is a prominent risk factor for many disabling and often fatal diseases, including stroke, myocardial infarction, arrhythmia, congestive heart failure, renal failure and retinopathy. It is estimated that 50 million Americans have high blood pressure.

Hypertension is classified as either essential hypertension or secondary hypertension. The former is the most common, with hypertension as the only symptom and no known underlying cause. Secondary hypertension is classified as hypertension with a known underlying medical condition responsible for the hypertensive state. The predisposing conditions may include renal disease, endocrine system abnormalities, adrenal or pituitary tumors, blood vessel irregularities, medications, and as recently discovered, genetic factors.

For the most part, the molecular pathogenesis and genetic factors of the most common forms of hypertension are poorly understood or unknown. However, the study of rare forms of hypertension in which the Mendelian inheritance patterns are better known may illuminate the causative genetics of this disease, and lead to wide ranging diagnostics and therapeutics that can be used to treat multiple forms of hypertension.

One such disease is pseudohypoaldersteronism type II (PHA II), an autosomal dominant disorder with clinical hallmarks of hypertension, hyperkalemia (increased serum potassium levels) and low or suppressed plasma renin activity. Genes implicated in PHA II have been mapped to chromosomes 17, 1, or 12, but no convincing results have indicated a specific gene or genes responsible for PHA II. The hypertension component of PHA II is attributed to increased renal salt reabsorption and the hyperkalemia to reduced renal potassium excretion despite normal glomerular filtration and aldosterone secretion. Further, reduced renal hydrogen ion secretion is often present, resulting in metabolic acidosis. PHA II is also known as chloride shunt syndrome because the symptoms are chloride dependent, and are ameliorated by substitution of sodium sulfate or sodium bicarbonate for sodium chloride. Treatment may also include thiazide diuretics, which inhibit salt reabsorption in the distal nephron. Overall, PHA II appears as a defect in renal electrolyte use, but with no readily explained physiological mechanisms.

PHA II, like other forms of hypertension, may be controlled with a rigorous diet and exercise regimen and treated with a lifetime of medication. A wide range of pharmaceutical treatments for hypertension exist. As previously mentioned, diuretics, including thiazide diuretics, are one option for the treatment of hypertension. Diuretics serve to increase urinary outflow, reducing renal salt and water retention, and thereby reducing blood pressure. Further, beta-blockers can be used to lower the heart rate and thereby lower the blood volume output. Angiotensin converting enzyme (ACE) II inhibitors block the production of ACE, a hormone that directs arterial constriction and indirectly promotes renal salt retention. Similarly, ACE receptor blockers prevent the action of ACE. Calcium channel blockers relax blood vessels to lower blood pressure. Alpha-1 blockers and Alpha-2 agonists block the effect of vessel constricting hormones such as norepinephrine and decrease central nervous system impulses that direct blood vessel constriction, respectively. Direct vasodilators work directly on blood vessels to relax the vessel wall, while sympathetic nerve blockers prevent peripheral nervous system mediated vessel constriction. Further, Rho kinase inhibitors have been proposed as therapeutics for hypertension (U.S. patent application Ser. No. 09/791,648). Rho kinase has been demonstrated to play a role in smooth muscle (blood vessel wall) constriction, and by specifically inhibiting Rho kinase, blood vessel walls relax.

While these methods described herein have been demonstrated to be effective, given that the Centers for Disease Control attributes almost 15,000 annual deaths in the U.S. solely due to hypertension, the present pharmaceutical means of treating high blood pressure are clearly not sufficient to address this significant health problem. Additionally, the aforementioned treatments are directed towards inhibiting or correcting the physiological mechanisms of this disease for a short time, and do not address the cause of the problem i.e., the molecular basis of hypertension. Therefore, while somewhat palliative, the prior art method used to treat hypertension fall far short of addressing this pressing health issue.

Given that almost a quarter of the world's population has high blood pressure, yet few of the molecular mechanisms underlying this disease are known, a long felt need exists to develop diagnostics, methods, and effective therapeutics for combating this disease. The present invention meets this need.

### BRIEF SUMMARY OF THE INVENTION

The invention includes an isolated nucleic acid encoding a mammalian WNK, wherein the nucleic acid shares greater than 86% identity with at least one of SEQ ID NO:1 and SEQ ID NO:3.

In one aspect, the nucleic acid further comprises a nucleic acid encoding a tag polypeptide covalently linked thereto.

The invention includes an isolated nucleic acid encoding human WNK, the nucleic acid having greater than 86% identity with a nucleic acid selected from the group consisting of SEQ ID NO:1 and SEQ ID NO:3.

The invention also includes an isolated nucleic acid encoding a mammalian WNK, wherein the nucleic acid is selected from the group consisting of an isolated nucleic acid consisting of the nucleotide sequence of SEQ ID NO:1, and an isolated nucleic acid consisting of the nucleotide sequence of SEQ ID NO:3.

The invention includes an isolated nucleic acid encoding a mammalian WNK, wherein the nucleic acid encodes human WNK1 and further wherein the nucleic acid shares greater than 86% identity with a nucleic acid having the nucleic acid sequence of SEQ ID NO:1, and further wherein the nucleic acid comprises a deletion of at least a portion of intron 1 of the genomic DNA encoding the human WNK1.

In one aspect, the deletion consists of a deletion from about nucleotide number 36018 to about nucleotide number 77314 relative to the sequence of BAC clone GenBank accession number AC004765.

In another aspect, the nucleic acid further comprises a nucleic acid specifying a promoter/regulatory sequence operably linked thereto.

In yet another aspect, the invention includes a vector comprising an isolated nucleic acid encoding a mammalian WNK, wherein the nucleic acid shares greater than 86% identity with at least one of SEQ ID NO:1 and SEQ ID NO:3.

In a further aspect, the invention includes a recombinant cell comprising an isolated nucleic acid encoding a mammalian WNK, wherein the nucleic acid shares greater than 86% identity with at least one of SEQ ID NO:1 and SEQ ID NO:3.

The invention includes an isolated nucleic acid complementary to an isolated nucleic acid encoding a mammalian WNK, or a fragment thereof, the complementary nucleic acid being in an antisense orientation.

In one aspect, the nucleic acid shares greater than 86% identity with a nucleic acid complementary with a nucleic acid having the sequence of at least one of a human WNK1 (SEQ ID NO:1), and a human WNK4 (SEQ ID NO:3).

The invention includes an isolated mammalian WNK polypeptide.

The invention includes an isolated mammalian WNK, wherein the WNK comprises an amino acid sequence having greater than 86% identity with a polypeptide having the amino acid sequence selected from the group consisting of an amino acid having the sequence of SEQ ID NO:2, and an amino acid having the sequence of SEQ ID NO:4.

The invention includes an isolated human WNK, wherein the WNK is selected from the group consisting of human WNK1 and human WNK4.

In one aspect, the human WNK is hWNK4 and further wherein the hWNK4 comprises a mutation selected from the group consisting of an amino acid substitution at amino acid residue number 565 from a glycine to a glutamine relative to the sequence of SEQ ID NO:4, an amino acid substitution at amino acid residue number 56 from aspartic acid to alanine relative to the sequence of SEQ ID NO:4, an amino acid substitution at amino acid residue number 562 from a glutamine to lysine relative to the sequence of SEQ ID NO:4, and an amino acid substitution at amino acid residue number 1185 from arginine to cysteine relative to the sequence of SEQ ID NO:4.

The invention includes an antibody that specifically binds with a mammalian WNK, or a fragment thereof.

In one aspect, the mammalian WNK shares greater than about 86% identity with a polypeptide having the amino acid sequence selected from the group consisting of SEQ ID NO:2 and SEQ ID NO:4.

In yet another aspect, the antibody is selected from the group consisting of a polyclonal antibody, a monoclonal antibody, and a synthetic antibody.

The invention includes a composition comprising an isolated nucleic acid encoding a mammalian WNK, wherein the nucleic acid shares greater than 86% identity with at least one of SEQ ID NO:1 and SEQ ID NO:3, and a pharmaceutically-acceptable carrier.

The invention includes a composition comprising an isolated mammalian WNK polypeptide and a pharmaceutically-acceptable carrier.

The invention includes a method of identifying a compound that inhibits expression of human WNK in a cell. The method comprises contacting a cell with a compound and comparing the level of expression of human WNK in the cell contacted with the compound with the level of expression of human WNK in an otherwise identical cell, wherein a lower level of expression of human WNK in the cell contacted with the compound compared with the level of expression of human WNK in the otherwise identical cell not contacted with the compound, is an indication that the compound inhibits expression of human WNK in the cell. In one aspect, the invention includes a compound identified by this method.

In another aspect, the human WNK is selected from the group consisting of hWNK1 and hWNK4.

The invention includes a method of treating a disease mediated by expression of a human WNK. The method comprises administering to a human patient afflicted with a disease mediated by expression of a human WNK, a human WNK expression-inhibiting amount of a WNK inhibitor, thereby treating a disease mediated by expression of a human WNK.

In one aspect, the disease is selected from the group consisting of hypertension and pseudohypoaldersteronism type II.

In another aspect, the disease is pseudohypoaldersteronism type II and further wherein the mammal is a human.

In yet another aspect, the WNK inhibitor comprises an isolated nucleic acid complementary to an isolated nucleic acid encoding a human WNK, or a fragment thereof, the complementary nucleic acid being in an antisense orientation.

The invention includes a method of treating hypertension in a mammal, wherein the hypertension is mediated by increased expression of a mammalian WNK1. The method comprises administering to a mammal afflicted with a disease mediated by increased expression of a mammalian WNK1, a WNK1 expression-inhibiting amount of a WNK inhibitor, thereby treating hypertension in the mammal.

The invention includes a method of treating pseudohypoaldersteronism type II in a mammal, wherein the pseudohypoaldersteronism type II is mediated by increased expression of a mammalian WNK1. The method comprises administering to a mammal afflicted with pseudohypoaldersteronism type II a WNK expression-inhibiting amount of a WNK inhibitor, thereby treating pseudohypoaldersteronism type II in the mammal.

The invention includes a method of treating hypertension in a mammal, wherein the hypertension is mediated by expression of a mutant mammalian WNK4. The method comprises administering to a mammal afflicted with a disease mediated by expression of a mutant mammalian WNK4, a WNK expression-inhibiting amount of a WNK inhibitor, thereby treating hypertension in the mammal.

The invention includes a method of treating pseudohypoaldersteronism type II in a mammal, wherein the pseudohypoaldersteronism type II is mediated by expression of a mutant mammalian WNK4. The method comprises administering to a mammal afflicted with pseudohypoaldersteronism type II, a WNK expression-inhibiting amount of a WNK inhibitor, thereby treating pseudohypoaldersteronism type II in the mammal.

The invention includes a method of identifying a human patient afflicted with a disease, disorder or condition associated with altered expression of WNK. The method comprises detecting the level of WNK expression in a human and comparing the level of expression of WNK in the human with the level of expression of WNK in a normal human not afflicted with a disease, disorder or condition associated with altered expression of WNK, thereby detecting a human patient afflicted with a disease, disorder or condition associated with altered expression of WNK.

In one aspect, the disease, disorder or condition associated with altered expression of WNK is selected from the group consisting of hypertension and pseudohypoaldersteronism type II.

The invention includes a method of detecting a mutation in a WNK allele in a human. The method comprises comparing the nucleic acid sequence encoding WNK of a human suspected of having a mutation in WNK with the nucleic acid sequence encoding WNK obtained from a normal human not having a mutation in WNK, wherein any difference between the nucleic acid sequence of the human suspected of having a mutation in WNK and the nucleic acid sequence encoding WNK of the normal human not having a mutation in WNK detects a mutation in a WNK allele in the human.

In one aspect, the WNK is human WNK4 and further wherein the mutation is selected from the group consisting of an amino acid substitution at amino acid residue number 565 from a glycine to a glutamine relative to the sequence of SEQ ID NO:4, an amino acid substitution at amino acid residue number 564 from aspartic acid to alanine relative to the sequence of SEQ ID NO:4, an amino acid substitution at amino acid residue number 562 from glutamine to lysine relative to the sequence of SEQ ID NO:4, and an amino acid substitution at amino acid residue number 1185 from arginine to cysteine relative to the sequence of SEQ ID NO:4.

The invention includes a method of detecting a mutation in a WNK allele in a human. The method comprises comparing the genomic nucleic acid sequence encoding WNK of a human suspected of having a mutation in WNK with the genomic nucleic acid sequence encoding WNK obtained from a normal human not having a mutation in WNK, wherein any difference between the genomic nucleic acid sequence of the human suspected of having a mutation in WNK and the genomic nucleic acid sequence encoding WNK of the normal human not having a mutation in WNK detects a mutation in a WNK allele in the human.

In one aspect, the WNK is hWNK1, and further wherein the mutation comprises deletion of at least a portion of intron 1.

In another aspect, the deletion consists of a deletion relative to the sequence of BAC clone GenBank accession number AC004765.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A and 1B, depicts the linkage of PHA II to the telomere of 12p in K22. FIG. 1A depicts the structure of PHA II kindred K22. Affected, unaffected, and deceased individuals of unknown phenotype are shown as filled, unfilled, and shaded symbols, respectively. Genotypes at loci from the telomeric segment of chromosome 12p are shown in their chromosomal order with the telomere at the top; estimated genetic distances between adjacent loci (in centimorgans, cM) are shown. The boxed haplotype cosegregates with the disease. Two independent recombination events in affected individuals define the location of the disease gene to the most telomeric 2-cM segment. In addition, four loci within this segment demonstrate hemizygosity in affected kindred members; the inferred null alleles are denoted as “0”. Genotypes that unambiguously demonstrate absence of transmission of an allele from affected parent to affected offspring are indicated by asterisks. FIG. 1B depicts the multipoint lod score for linkage of PHA II to 12p in K22. The map of marker loci used in the multipoint analysis is shown at the top of the figure, and the 1000:1 support interval for the PHA IIC locus is indicated by the thick bar.

FIG. 2, comprising FIG. 2A through 2G, depicts characterization of deletions in WNK1 in PHA II. FIG. 2A depicts the structure of wild-type (WT) and deleted alleles on 12p in K22. Polymorphic sequence tagged sites (STSs) are indicated above the horizontal line representing genomic segments, and cleavage sites for Pvull are indicated below. The locations of probes used for Southern blotting in (B) and the sizes of resulting fragments are indicated. FIG. 2B depicts the identification of deletion endpoints by Southern blotting in K22. Southern blots hybridizing probes from (A) to genomic DNA digested with Pvull are shown. Affected individuals of K22 are indicated by asterisks. FIG. 2C depicts a PCR reaction across deletion endpoints in kindred K22. Products of PCR using primers separated by 42 kb in normal genomic DNA are shown for members of K22 (M denotes marker lane); a 600-bp fragment cosegregates with PHAII. Below, the DNA sequence of a portion of the PCR product is compared to the sequence of the wild-type segment (WT). Numbered bases correspond to positions on the BAC clone in GenBank accession AC004765. The PCR product arises from a deletion whose endpoints fuse sequences normally separated by 41 kb. The deletion endpoints occur within a 32-bp repeated sequence (underlined). FIG. 2D depicts the genomic structure of hWNK1. The genomic segment spanning WNK1 is represented by a horizontal line, and exons are indicated by numbered vertical lines. The genomic segments that are deleted in K22 and K4 are indicated. FIG. 2E depicts a Northern blot of hWNK1. A probe from hWNK1 was hybridized to RNA from a variety of human tissues. Locations of size standards in kilobases are indicated. FIG. 2F depicts a PCR reaction across deletion endpoints in PHA II kindred K4. Primers separated by 24 kb in normal genomic DNA directed PCR from genomic DNA of members of K4; a 2.4-kb product cosegregates with PHA II. The DNA sequence demonstrated a 21.8-kb deletion, with the deletion endpoints in a 16-bp repeated sequence (underlined). FIG. 2G depicts increased levels of hWNK1 transcripts in PHA II. Quantitative RT-PCR was used to compare the levels of hWNK1 and GAPDH transcripts in leukocytes from affected members of K4 (filled symbols) and control subjects (two unaffected members of K4 and one normal control subject; unfilled symbols). The mean and extreme values of repeated measures of the ratio of WNK1:GAPDH for each subject are shown.

FIG. 3, comprising FIG. 3A through 3C, depicts characterization of hWNK4. FIG. 3A depicts genomic structure of hWNK4. The genomic segment spanning WNK4 is represented by a horizontal line, and exons of the gene are indicated by numbered vertical lines. FIG. 3B depicts a comparison of hWNK1 and hWNK4. Domains of each protein are shown and the percentage amino acid identity (ID) between similar segments is indicated. Putative coil domains were predicted using the COILS program. FIG. 3C depicts a Northern blot of hWNK4. A probe from hWNK4 was hybridized to RNA from a variety of human tissues. Locations of size standards in kilobases are indicated.

FIG. 4, comprising FIG. 4A through 4F, depicts missense mutations in hWNK4 in PHA II. FIG. 4A depicts a mutation in exon 7 of hWNK4 that segregates with PHA II in K13. Products of SSCP from exon 7 are shown in members of K13. A novel variant (indicated by arrow) cosegregates with PHA II. FIG. 2B depicts mutations in exon 7 of hWNK4 in PHA II kindreds K11 and K23. Exon 7 was amplified as in FIG. 4A. Affected members are indicated by asterisks and show novel variants (arrows) not seen in normal subjects (N). FIG. 4C depicts the DNA sequence of mutations in exon 7. The top panel shows the wild-type (WT) DNA sequence for codons 560 through 566 of hWNK4; the encoded amino acid sequence is shown above. In lower panels, the sequences of the variants identified in FIG. 4A and FIG. 4B are shown. Mutations are indicated by asterisks and the altered amino acids are shown in red. FIG. 4D depicts a mutation in exon 17 in PHA II kindred K21. Exon 17 was amplified and fractionated as in panel FIG. 4A. The three affected members of K21 (asterisks) show a novel variant (arrow). FIG. 4E depicts the DNA sequence of the mutation in exon 17. The WT DNA sequence for codons 1182 through 1188 of hWNK4 is shown at the top, and the mutant sequence in K21 is shown below. FIG. 4F depicts conservation of residues mutated in PHA II among WNK family members. An 18 amino acid sequence of paralogous segments of hWNK1-4 is shown. An acidic 10 amino acid segment is highly conserved among all WNK family members. The mutations found in PHA II kindreds alter completely conserved residues.

FIG. 5, comprising FIGS. 5A through 5E, depicts localization of WNK1 in kidney. Frozen mouse kidney sections were stained with antibodies and analyzed by fluorescence microscopy. FIG. 5A depicts a low-power view of renal cortex stained with anti-WNK1 and anti-aquaporin-2 (AQP2), a marker of the connecting tubule and collecting duct. All tubules staining for AQP2 also stain for WNK1. In addition, other tubules in the cortex are also stained (DCT, see below). FIG. 5B depicts a transverse section of the cortical collecting duct (CCD), showing co-staining with anti-WNK1 and anti-AQP2. FIG. 5C depicts the same view as FIG. 5B, showing only anti-WNK1 channel, and demonstrating cytoplasmic distribution of WNK1. FIG. 5D depicts the transverse section of a distal convoluted tubule (DCT ) stained with anti-WNK1 and an antibody to the thiazide-sensitive sodium chloride cotransporter (NCCT) an apical marker of the DCT. All tubules staining for NCCT also stain for WNK1. FIG. 5E depicts the same view as FIG. 5D showing only anti-WNK1 channel. White bars represent 10 μm.

FIG. 6, comprising FIGS. 6A through 6H, depict localization of WNK4 in kidney. Frozen mouse kidney sections were stained with anti-bodies as in FIG. 5. FIG. 6A depicts low-power view of renal cortex stained with anti-WNK4 and anti-AQP2. Segments staining for AQP2 also stain for WNK4; other tubules in the cortex (DCT) are also stained by anti-WNK4. FIG. 2B depicts the cortical collecting duct (CCD) showing staining with both anti-WNK4 and anti-AQP2. FIG. 6C depicts the same view as FIG. 6B, showing only the WNK4 staining. WNK4 localizes to both intercellular junctions and the cytoplasm in the CCD. FIG. 6D depicts the distal convoluted tubule (DCT) showings staining with both anti-WNK4 and anti-NCCT. FIG. 6E depicts the same view as FIG. 6D, showing only WNK4 staining. WNK4 localizes virtually exclusively to intercellular junctions of the DCT. FIG. 6F through 6G depict colocalization of WNK4 with ZO-1, a tight junction protein. Sections were stained with antibodies to WNK4 and ZO-1. The WNK4 signal is shown alone in FIG. 6F, ZO-1 alone in FIG. 6G, and the two are superimposed in FIG. 6H. The two proteins colocalize, demonstrating that WNK4 is associated with the tight junctional complex. Bars, 10 μm.

### DETAILED DESCRIPTION OF THE INVENTION

The present invention relates to the novel discovery that two genes, WNK1 and WNK4 (with no lysine (K)), both serine/threonine kinases, are responsible for a form of secondary hypertension, PHA II. Briefly, and as described more fully herein, deletions in the genomic DNA encoding WNK1 lead to increased expression of the transcript, which is correlated with hypertension, and missense mutations concentrated in a highly conserved domain of WNK4 strongly suggest a loss of normal regulation, also correlated with hypertension. Both mutations are therefore indicative of a gain-of-function phenotype in the closely regulated balance between salt reabsorption, ion transport and gradient formation in the nephron, leading to hypertension and the additional symptoms of PHA II.

The symptoms of PHA II are strikingly similar to the prominent manifestations of essential, or common, hypertension. As an example, essential hypertension, like PHA II, is often characterized by low plasma renin activity and by a positive response to thiazide diuretics. Thus, given the similarities between the symptoms of PHA II and those of essential hypertension, treating PHA II can be an effective treatment of hypertension in general. Additionally, as the link between WNK kinases and hypertension is a novel finding, the data disclosed herein demonstrate that mutations and abnormal function in WNK can mediate forms of essential hypertension, such that the present invention provides novel therapeutics and diagnostics relating thereto.

Definitions

The articles “a” and “an” are used herein to refer to one or to more than one (i.e. to at least one) of the grammatical object of the article. By way of example, “an element” means one element or more than one element.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

“Altered expression” is used herein to refer to a higher, lower or otherwise different level of expression as compared to the level of expression exhibited in under normal circumstances.

As used herein, the term “antisense oligonucleotide” means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

The terms “complementary” and “antisense” as used herein, are not entirely synonymous. “Antisense” refers particularly to the nucleic acid sequence of the non-coding strand of a double stranded DNA molecule encoding a protein, or to a sequence which is substantially homologous to the non-coding strand. “Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

“Amplification” as used herein refers to any means by which a polynucleotide sequence is copied and thus expanded into a larger number of polynucleotide molecules, e.g., by reverse transcription, polymerase chain reaction, and ligase chain reaction.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs).

“Encoding” refers to the inherent property of specific sequences of nucleotides in a polynucleotide, such as a gene, a genomic DNA, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “functional” biological molecule is a biological molecule in a form in which it exhibits a property by which it is characterized. A functional enzyme, for example, is one which exhibits the characteristic catalytic activity by which the enzyme is characterized.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 1000 nucleotides, yet even more preferably, at least about 1000 to about 2380, more preferably, at least about 2380 nucleotides to about 3500 nucleotides, even more preferably, at least about 3500 nucleotides to about 5000 nucleotides, yet even more preferably, at least about 5000 to about 6000, even more preferably, at least about 6000 nucleotides to about 7000 nucleotides, yet even more preferably, at least about 7000 to about 7149, and most preferably, the nucleic acid fragment will be greater than about 7149 nucleotides in length.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

As applied to a protein, a “fragment” of WNK is about 20 amino acids in length. More preferably, the fragment of a WNK is about 30 amino acids, even more preferably, at least about 40, yet more preferably, at least about 60, even more preferably, at least about 80, yet more preferably, at least about 100, even more preferably, about 200, even more preferably, at least about 500, yet more preferably, at least about 1000, even more preferably, at least about 1240, more preferably, at least about 1350, even more preferably, at least about 1500, yet more preferably, at least about 2000, even more preferably, about 2300, and more preferably, at least about 2380 amino acids in length.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

In addition, when the terms “homology” or “identity” are used herein to refer to the nucleic acids and proteins, it should be construed to be applied to homology or identity at both the nucleic acid and the amino acid sequence levels.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example, at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein.

To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

A “portion” of a polynucleotide means at least at least about twenty sequential nucleotide residues of the polynucleotide. It is understood that a portion of a polynucleotide may include every nucleotide residue of the polynucleotide.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, the term “WNK” means any without lysine (K) kinase molecule having WNK activity, and having at least 66% amino acid identity with at least one of WNK1 and WNK4 (SEQ ID NOS: 2 and 4, respectively) as defined herein.

By the term “WNK activity,” as used herein, is meant the activity of a kinase to, among other things, phosphorylate serine and threonine, increase blood pressure, increase renal salt absorption, increase serum potassium level, reduce renal hydrogen ion secretion, decrease renal electrolyte handling, and the like.

“WNK expression-inhibiting amount,” as used herein, means any amount of a substance or molecule that detectably decreases the level of WNK expression, amount, and/or activity compared with the level of WNK expression, amount, and/or activity in the absence of the substance or molecule. Thus, any amount that mediates a detectable decrease in: the amount of WNK present, the level of WNK mRNA expression, and/or the ability of WNK to form necessary ligand/receptor interactions, is encompassed in the present invention. The assays by which these conditions are examined are well-known in the art and several are exemplified herein.

A “restriction site” is a portion of a double-stranded nucleic acid which is recognized by a restriction endonuclease.

By the term “specifically binds,” as used herein, is meant a compound, e.g., a protein, a nucleic acid, an antibody, and the like, which recognizes and binds a specific molecule, but does not substantially recognize or bind other molecules in a sample.

As used herein, the term “transgene” means an exogenous nucleic acid sequence which exogenous nucleic acid is encoded by a transgenic cell or mammal.

A “recombinant cell” is a cell that comprises a transgene. Such a cell may be a eukaryotic cell or a prokaryotic cell. Also, the transgenic cell encompasses, but is not limited to, an embryonic stem cell comprising the transgene, a cell obtained from a chimeric mammal derived from a transgenic ES cell where the cell comprises the transgene, a cell obtained from a transgenic mammal, or fetal or placental tissue thereof, and a prokaryotic cell comprising the transgene.

By “tag” polypeptide is meant any protein which, when linked by a peptide bond to a protein of interest, may be used to localize the protein, to purify it from a cell extract, to immobilize it for use in binding assays, or to otherwise study its biological properties and/or function.

As used herein, to “treat” means reducing the frequency with which symptoms of hypertension, PHA II, hyperkalemia, defective renal electrolyte handling, and the like, are experienced by a patient.

As used herein, to “alleviating” means reducing the severity with which symptoms of hypertension, PHA II, hyperkalemia, defective renal electrolyte handling, among other symptoms, are experienced by a patient. The skilled artisan, armed with the teachings provided herein, would understand what is meant by the symptoms of hypertension and PHA II, and what is encompassed thereby.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “WNK inhibitor” is used herein to refer to a composition of matter that prevents the biological function or expression of a WNK kinase. Such inhibitors include, but are not limited to, an antibody, a small molecule, a chemical compound, a peptidomimetic, a protein, a peptide, a nucleic acid, a ribozyme, and an antisense nucleic acid.

Description

I. Isolated Nucleic Acids

A. Sense Nucleic Acids

The present invention includes an isolated nucleic acid encoding a mammalian WNK molecule, or a fragment thereof, wherein the nucleic acid shares at least about 66% identity with at least one nucleic acid having the sequence of SEQ ID NO:1 and SEQ ID NO:3. Preferably, the nucleic acid is about 70% homologous, more preferably, about 80% homologous, more preferably, about 90% homologous, even more preferably, about 95% homologous, and most preferably, about 99% homologous to at least one of SEQ ID NO:1 and SEQ ID NO:3, disclosed herein. Even more preferably, the nucleic acid is at least one of SEQ ID NO:1 and SEQ ID NO:3.

The present invention includes an isolated nucleic acid encoding human WNK(hWNK1), or a fragment thereof, wherein the nucleic acid shares greater than about 86% homology with (hWNK1) SEQ ID NO:1. Preferably, the nucleic acid is about 87% homologous, more preferably, about 90% homologous, even more preferably, about 95% homologous, and most preferably, about 99% homologous to the hWNK1 disclosed herein, SEQ ID NO:1. Even more preferably, the nucleic acid is SEQ ID NO:1.

The present invention includes an isolated nucleic acid encoding human WNK4 (hWNK4), or a fragment thereof, wherein the nucleic acid shares greater than about 86% homology with (hWNK4) SEQ ID NO:3. Preferably, the nucleic acid is about 87% homologous, more preferably, about 90% homologous, even more preferably, about 95% homologous, and most preferably, about 99% homologous to the hWNK4 disclosed herein, SEQ ID NO:3. Even more preferably, the nucleic acid is SEQ ID NO:3.

In another aspect, the present invention includes an isolated nucleic acid encoding a human WNK molecule, or a fragment thereof, wherein the protein encoded by the nucleic acid shares greater than about 86% homology with the amino acid sequence of at least one of SEQ ID NO:2 and SEQ ID NO:4. Preferably, the nucleic acid is about 87% homologous, more preferably, about 90% homologous, even more preferably, about 95% homologous, and most preferably, about 99% homologous to at least one of SEQ ID NO:2 and SEQ ID NO:4. Even more preferably, the human WNK molecule protein encoded by the nucleic acid is at least one of SEQ ID NO:2 and SEQ ID NO:4.

In another aspect, the present invention includes an isolated nucleic acid encoding human WNK1 protein, or a fragment thereof, wherein the protein encoded by the nucleic acid shares greater than about 86% homology with the amino acid sequence of SEQ ID NO:2. Preferably, the protein encoded by the nucleic acid is about 87% homologous, more preferably, about 90% homologous, even more preferably, about 95% homologous, and most preferably, about 99% homologous to the hWNK1 disclosed herein, SEQ ID NO:2. Even more preferably, the hWNK1 protein encoded by the nucleic acid is SEQ ID NO:2.

In another aspect, the present invention includes an isolated nucleic acid encoding human WNK4 (hWNK4), or a fragment thereof, wherein the protein encoded by the nucleic acid shares greater than about 86% homology with the amino acid sequence of SEQ ID NO:4. Preferably, the protein encoded by the nucleic acid is about 87% homologous, more preferably, about 90% homologous, even more preferably, about 95% homologous, and most preferably, about 99% homologous to the hWNK4 disclosed herein, SEQ ID NO:4. Even more preferably, the hWNK4 protein encoded by the nucleic acid is SEQ ID NO:4.

One skilled in the art would appreciate, based upon the disclosure provided herein, that a human WNK homolog likely exists and can be readily identified and isolated using the methods described herein using the sequence data disclosed herein regarding the highly-conserved rat and human homologs. Thus, the present invention encompasses additional WNKs that can be readily identified based upon the disclosure provided herein, including, but not limited to, human WNK.

The isolated nucleic acid of the invention should be construed to include an RNA or a DNA sequence encoding a WNK protein of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

The present invention should not be construed as being limited solely to the nucleic and amino acid sequences disclosed herein. Once armed with the present invention, it is readily apparent to one skilled in the art that other nucleic acids encoding WNK proteins can such as those present in other species of mammals (e.g., ape, gibbon, bovine, ovine, equine, porcine, canine, feline, and the like) be obtained by following the procedures described herein in the experimental details section for the isolation of the rat, and human WNK nucleic acids encoding WNK polypeptides as disclosed herein (e.g., screening of genomic or cDNA libraries), and procedures that are well-known in the art (e.g., reverse transcription PCR using mRNA samples) or to be developed.

One of skill in the art, when armed with this disclosure and the data disclosed herein, will readily understand that the present invention encompasses mammalian WNK nucleic acid molecules comprising mutations in the genomic DNA. For example, and as more fully discussed elsewhere herein, a deletion in intron 1 of the genomic DNA encoding mammalian WNK1 leads to, among other things, increased expression of the transcript, resulting in hypertension, PHA II, hyperkalemia, and the like. As further exemplified herein, the deletion can occur in the genomic DNA encoding WNK. Preferably, the delection consists of a deletion from about nucleotide number 36018 to about nucleotide number 77314, relative to the nucleic acid of BAC clone GenBank Accession No. AC004765. However, the skilled artisan will also appreciate that the present invention is in no way limited to this specific deletion, as mammalian WNK1 is encoded from twenty-eight exons spanning about 156 kilobases of genomic DNA. Thereby, the skilled artisan, when equipped with the present disclosure, can easily identify other mutations leading to altered expression of mammalian WNK1, and therefore other mutations associated with, among other things, hypertension, PHA II, defects in renal electrolyte handling, and the like.

The skilled artisan, when armed with the present disclosure and the data incorporated herein, will further understand that other mutations in the genomic DNA encoding WNK1 can lead to altered expression, and therefore hypertension, PHA II, defects in renal electrolyte handling, and the like. As detailed in the data disclosed herein, many methods exist for the identification and characterization of deletions in the genomic DNA encoding mammalian WNK1, including, but not limited to, Southern blotting, PCR, and other methods well known in the art for identifying deletions in a nucleic acid molecule. Thereby, the present invention encompasses mutations, known or to be discovered, in the genomic DNA encoding mammalian WNK1.

Additionally, one of skill in the art will understand, based on the present disclosure and teachings provided herein, that missense mutations in the nucleic acid sequence encoding mammalian WNK4 lead to, among other things, hypertension, PHA II, decreased salt reabsorption, hyperkalemia, and the like. As detailed elsewhere herein, missense mutations in the nucleic acid encoding portions of mammalian WNK4 just distal to the first and second putative coil domains, result in mutations in the highly conserved regions of mammalian WNK4. The skilled artisan will also appreciate that the present invention is in no way limited to these specific missense mutations. For instance, as these mutations were discovered in highly conserved domains, the routineer would appreciate once armed with these teachings, that additional mutations in these same domains can be useful. Thereby, the skilled artisan, when equipped with the present disclosure, can easily identify other mutations, including, but not limited to, missense mutations, leading to altered expression and/or function of mammalian WNK, preferably WNK4. Therefore other mutations associated with, among other things, hypertension, PHA II, defects in renal electrolyte handling, and the like can be identified according to the teachings set forth herein and the present invention is not limited solely to these mutations disclosed herein.

For instance, the skilled artisan, when armed with the present disclosure and the data incorporated herein, will further understand that other mutations in the DNA encoding WNK4 can lead to altered expression and/ or function, and therefore hypertension, PHA II, defects in renal electrolyte handling, and the like. As detailed elsewhere herein, many methods exist for the identification and characterization of missense or other mutations in the nucleic acid encoding mammalian WNK4. Such methods include, but are not limited to, expressing putative mutated nucleic acids in recombinant cells or transgenic animals, and looking for manifestations of altered expression and/or function, including, but not limited to, hyperkalemia, increased renal salt reabsorption, reduced hydrogen ion secretion, metabolic acidosis, suppressed plasma renin activity, and the like. Thereby, the present invention encompasses mutations, known or to be discovered, in mammalian WNK4, especially since two specific regions have been identified herein as sites of potentially useful mutations.

Further, any number of procedures may be used for the generation of mutant, derivative or variant forms of WNK using recombinant DNA methodology well known in the art such as, for example, that described in Sambrook et al. (1989, Molecular Cloning: A Laboratory Manual, Cold Spring Harbor Laboratory Press, New York) and Ausubel et al. (1997, Current Protocols in Molecular Biology, Green & Wiley, New York).

Procedures for the introduction of amino acid changes in a protein or polypeptide by altering the DNA sequence encoding the polypeptide are well known in the art and are also described in Sambrook et al. (1989, Molecular Cloning: A Laboratory Manual, Cold Spring Harbor Laboratory Press, New York); Ausubel et al. (1997, Current Protocols in Molecular Biology, Green & Wiley, New York).

The invention includes a nucleic acid encoding a mammalian WNK wherein the nucleic acid encoding a tag polypeptide is covalently linked thereto. That is, the invention encompasses a chimeric nucleic acid wherein the nucleic acid sequences encoding a tag polypeptide is covalently linked to the nucleic acid encoding at least one of human WNK1 and human WNK4. Such tag polypeptides are well known in the art and include, for instance, green fluorescent protein (GFP), myc, myc-pyruvate kinase (myc-PK), His₆, maltose biding protein (MBP), an influenza virus hemagglutinin tag polypeptide, a flag tag polypeptide (FLAG), and a glutathione-S-transferase (GST) tag polypeptide. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention.

The nucleic acid comprising a nucleic acid encoding a tag polypeptide can be used to localize WNK within a cell, a tissue, and/or a whole organism (e.g., a mammalian embryo), detect WNK secreted from a cell, and to study the role(s) of WNK in a cell or animal. Further, addition of a tag polypeptide facilitates isolation and purification of the “tagged” protein such that the proteins of the invention can be produced and purified readily.

B. Antisense Nucleic Acids and Ribozymes

In certain situations, it may be desirable to inhibit expression of WNK and the invention therefore includes compositions useful for inhibition of WNK expression. Thus, the invention features an isolated nucleic acid complementary to a portion or all of a nucleic acid encoding a mammalian WNK molecule which nucleic acid is in an antisense orientation with respect to transcription. Preferably, the antisense nucleic acid is complementary with a nucleic acid having greater than about 86% homology with at least one of SEQ ID NO:1 and SEQ ID NO:3, or a fragment thereof. Preferably, the nucleic acid is about 87% homologous, even more preferably, about 90% homologous, and most preferably, about 95% homologous to a nucleic acid complementary to a portion or all of a nucleic acid encoding a mammalian WNK having the sequence of at least one of SEQ ID NO:1 and SEQ ID NO:3, or a fragment thereof, which is in an antisense orientation with respect to transcription. Most preferably, the nucleic acid is complementary to a portion or all of a nucleic acid that is at least one of SEQ ID NO:1 and SEQ ID NO:3, or a fragment thereof. Such antisense nucleic acid serves to inhibit the expression, function, or both, of a WNK molecule.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (Cohen, 1989, In: Oligodeoxyribonucleotides, Antisense Inhibitors of Gene Expression, CRC Press, Boca Raton, Fla.; Tullis, 1991, U.S. Pat. No. 5,023,243, incorporated by reference herein in its entirety).

Further, the invention includes a recombinant cell comprising an antisense nucleic acid which cell is a useful model for elucidating the role(s) of WNK in cellular processes. That is, the increased expression of WNK in humans indicates that WNK is involved in hypertension, high serum potassium levels, reduced renal potassium excretion, reduced renal hydrogen ion secretion, and the like. Accordingly, a transgenic cell comprising an antisense nucleic acid complementary to WNK is a useful tool for the study of the mechanism(s) of action of WNK and its role(s) in the cell and for the identification of therapeutics that ameliorate the effect(s) of WNK expression. Further, methods of decreasing WNK expression and/or activity in a cell can provide useful diagnostics and/or therapeutics for diseases, disorders or conditions mediated by or associated with increased WNK expression, increased level of WNK protein in a cell or secretion therefrom, and/or increased WNK activity. Such diseases, disorders or conditions include, but are not limited to, hypertension, PHA II, renal disease, and the like, which are mediated by or associated with increased WNK1 and/or WNK4 expression.

One skilled in the art will appreciate that one way to decrease the levels of WNK mRNA and/or protein in a cell is to inhibit expression of the nucleic acid encoding the protein. Expression of WNK may be inhibited using, for example, antisense molecules, and also by using ribozymes or double-stranded RNA as described in, for example, Wianny and Kernicka-Goetz (2000, Nature Cell Biol. 2:70-75).

Antisense molecules and their use for inhibiting gene expression are well known in the art (see, e.g., Cohen, 1989, In: Oligodeoxyribonucleotides, Antisense Inhibitors of Gene Expression, CRC Press, Boca Raton, Fla.). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue (1993, U.S. Pat. No. 5,190,931).

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see Cohen, In: Oligodeoxyribonucleotides, Antisense Inhibitors of Gene Expression, CRC Press, Boca Raton, Fla.; Tullis, 1991, U.S. Pat. No. 5,023,243, incorporated by reference herein in its entirety).

Ribozymes and their use for inhibiting gene expression are also well known in the art (Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053, incorporated by reference herein in its entirety). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is that, because they are sequence-specific, only mRNAs with particular sequences are inactivated.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of WNK may be designed by incorporating target sequences into the basic ribozyme structure which are complementary to the mRNA sequence of the WNK encoded by WNK or having at least about 80% homology to at least one of SEQ ID NO:1 and SEQ ID NO:8. Ribozymes targeting WNK may be synthesized using commercially available reagents (Applied Biosystems, Inc., Foster City, Calif.) or they may be genetically expressed from DNA encoding them.

II. Isolated Polypeptides

The invention also includes an isolated polypeptide comprising a mammalian WNK molecule. Preferably, the isolated polypeptide comprising a mammalian WNK molecule is greater than about 86% homologous to a polypeptide having the amino acid sequence of at least one of SEQ ID NO:2 and SEQ ID NO:4. The skilled artisan would understand, based upon the disclosure provided herein, that the polypeptide of the invention does not include rat WNK1, described in Xu et al., 2000, J. Biol. Chem. 275:16795. Preferably, the isolated polypeptide is about 87% homologous, more preferably, about 90% homologous, even more preferably, about 95% homologous, and most preferably, about 99% homologous to at least one of SEQ ID NO:2 and SEQ ID NO:4. More preferably, the isolated polypeptide comprising a mammalian WNK is at least one of human WNK1 and WNK4. Most preferably, the isolated polypeptide comprising a mammalian WNK molecule is at least one of SEQ ID NO:2 and SEQ ID NO:4.

The invention also includes an isolated polypeptide comprising a mammalian WNK molecule. Preferably, the isolated polypeptide comprising a mammalian WNK is greater than about 86% homologous to a polypeptide having the amino acid sequence of SEQ ID NO:2. More preferably, the isolated polypeptide comprising a human WNK1 is at least about 87%, more preferably, about 90% homologous, even more preferably, about 95% homologous, and more preferably, at least about 99% homologous to human WNK1. More preferably, the isolated polypeptide comprising a mammalian WNK molecule is human WNK1. Most preferably, the isolated polypeptide comprising a mammalian WNK molecule is SEQ ID NO:2.

The invention also includes an isolated polypeptide comprising a mammalian WNK molecule. Preferably, the isolated polypeptide comprising a mammalian WNK is greater than about 86% homologous to a polypeptide having the amino acid sequence of SEQ ID NO:4. More preferably, the isolated polypeptide comprising a human WNK4 is at least about 87%, more preferably, about 90% homologous, even more preferably, about 95% homologous, and more preferably, at least about 99% homologous to human WNK4. More preferably, the isolated polypeptide comprising a mammalian WNK molecule is human WNK4. Most preferably, the isolated polypeptide comprising a mammalian WNK molecule is SEQ ID NO:4.

The present invention also provides for analogs of proteins or peptides which comprise a WNK molecule as disclosed herein. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Additionally, one of skill in the art will understand, based on the present disclosure and teachings provided herein, that substitutions in the amino acid sequence of mammalian WNK4 lead to, among other things, hypertension, hyperkalamia, PHA II, decreased salt reabsorption, and the like. As detailed elsewhere herein, amino acid substitutions in the amino acid sequence of mammalian WNK4 just distal to the first and second putative coil domains, specifically a substitution of Gln⁵⁶⁵ for Glu, a substitution of Asp⁵⁶⁴ for Ala, a substitution of Glu⁵⁶² for Lys, and a substitution of Arg¹¹⁸⁵ for Cys, result in charge-changing substitutions in the conserved regions of mammalian WNK4. The skilled artisan will also appreciate that the present invention is in no way limited to these specific substitutions. For instance, as these substitutions were discovered in highly conserved domains, additional substitutions in these and other domains can be used to identify additional potential useful mutations. Thereby, the skilled artisan, when equipped with the present disclosure, can readily identify other substitutions, including, but not limited to, conservative substitutions, charge-changing substitutions, and the like, leading to altered expression and/or function of mammalian WNK, preferably, but not limited to, WNK4, and therefore other substitutions associated with, among other things, hypertension, PHA II, defects in renal electrolyte handling, and the like are encompassed in the present invention.

The skilled artisan, when armed with the present disclosure and the data disclosed herein, will further understand that other substitutions in the amino acid sequence of WNK4 can lead to altered expression and/or function, and therefore hypertension, PHA II, defects in renal electrolyte handling, and the like. As detailed elsewhere herein, many methods are well known in the art for the identification and characterization of substitutions in the amino acid sequence of mammalian WNK4. Thereby, the present invention encompasses amino acid substitutions, known or to be discovered, in mammalian WNK, preferably WNK4.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The present invention should also be construed to encompass “mutants,” “derivatives,” and “variants” of the peptides of the invention (or of the DNA encoding the same) which mutants, derivatives and variants are WNK peptides which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of the WNK peptide of the present invention (e.g., a substitution of Gln⁵⁶⁵ for Glu, a substitution of Asp⁵⁶⁴ for Ala, a substitution of Glu⁵⁶² for Lys, and a substitution of Arg¹¹⁸⁵ for Cys).

A biological property of a WNK protein should be construed but not be limited to include, the ability of the peptide to be present in a cell, to act locally or via circulating in the bloodstream or in other body fluids, to function in the tight junctions and other areas of the kidney, including, but not limited to the cortical collecting duct, the distal convoluted tubule, and the medullary collecting duct, and the like.

The skilled artisan would understand, based upon the disclosure provided herein, that WNK biological activity encompasses, but is not limited to, the ability of a molecule or compound to be expressed in kidney tissue, to be detected in kidney cells, to be expressed in a cell, and the like. “WNK activity” includes the effects of WNK, either that expressed in the kidney, or that found in other parts of the body. WNK biological activity mediates, is associated with, or both, inter alia, hypertension, pseudohypoaldosteronism type II, ion flux management in the renal system, and the like.

One skilled in the art would also appreciate, based on the disclosure provided herein, that WNK biological activity includes, but is not limited to, to increase serum potassium levels, the ability to mediate renal ion flux, salt flux, and water flux, pH homeostasis, to be present in a cell of the renal system or other organs, to be expressed in the mammalian renal system, to be present in the cytoplasm and/or intercellular junctions, to be present in the tight junctions, kinase activity, and the like.

Further, the invention should be construed to include naturally occurring variants or recombinantly derived mutants of WNK sequences, which variants or mutants render the protein encoded thereby either more, less, or just as biologically active as the full-length clones of the invention.

The nucleic acids, and peptides encoded thereby, are useful tools for elucidating the function(s) of WNK molecule in a cell. Further, nucleic and amino acids comprising mammalian WNK molecule are useful diagnostics which can be used, for example, to identify a compound that affects WNK expression or expression of WNK mutants, which compound is a potential hypertension or other renal-associated disease drug candidate. The nucleic acids, the proteins encoded thereby, or both, can be administered to a mammal to increase or decrease expression of WNK in the mammal. This can be beneficial for the mammal in situations where under or over-expression of WNK1 and/or WNK4 in the mammal mediates a disease or condition associated with altered expression of WNK compared with normal expression of WNK in a healthy mammal.

That is, the data disclosed herein demonstrate that malexpression of WNK is associated with hypertension and PHA II. Further, the data disclosed herein demonstrate, for the first time, that malexpression of WNK is associated with inter alia, hypertension and PHA II, renal disease, and the like, such that affecting expression of WNK has an effect on such conditions.

Additionally, the nucleic and amino acids of the invention can be used to produce recombinant cells and transgenic non-human mammals which are useful tools for the study of WNK action, the identification of novel diagnostics and therapeutics for treatment of hypertension, PHA II, renal disease, and for elucidating the cellular role(s) of WNKs, among other things. Further, the nucleic and amino acids of the invention can be used diagnostically, either by assessing the level of gene expression or protein expression, to assess severity and prognosis of hypertension and PHA II. The nucleic acids and proteins of the invention are also useful in the development of assays to assess the efficacy of a treatment for hypertension, PHA II, and renal diseases or disorders. That is, the nucleic acids and polypeptides of the invention can be used to detect the effect of various therapies on WNK molecule expression, thereby ascertaining the effectiveness of the therapies. The nucleic acids and proteins of the present invention are also useful to detect mutations that are correlated with hypertension. This is because, as disclosed herein, mutations in the first intron of the genomic DNA encoding mammalian WNK1, and amino acid substitutions in two conserved regions of mammalian WNK4 result in hypertension, PHA II, hyperkalemia, and the like. Thereby, the nucleic acids and proteins of the present invention can provide useful diagnostic tools for, among other things, hypertension.

III. Vectors

In other related aspects, the invention includes an isolated nucleic acid encoding a mammalian WNK operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

Expression of WNK, either alone or fused to a detectable tag polypeptide, in cells which either do not normally express the WNK or which do not express WNK fused with a tag polypeptide, may be accomplished by generating a plasmid, viral, or other type of vector comprising the desired nucleic acid operably linked to a promoter/regulatory sequence which serves to drive expression of the protein, with or without tag, in cells in which the vector is introduced. Many promoter/regulatory sequences useful for driving constitutive expression of a gene are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of the nucleic acid encoding WNK may be accomplished by placing the nucleic acid encoding WNK, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Expressing WNK using a vector allows the isolation of large amounts of recombinantly produced protein. Further, where the increased level of WNK expression caused by mutations in the WNK nucleic acid sequence results in a disease, disorder, or condition associated with such expression, the expression of WNK driven by a promoter/regulatory sequence can provide useful therapeutics including, but not limited to, gene therapy whereby WNK is provided. A disease, disorder or condition associated with an increased level of expression, level of protein, or increased activity of the protein, for which administration of WNK can be useful can include, but is not limited to, hypertension, PHA II, renal diseases and disorders, and the like. Preferably, a disease, disorder or condition associated with an increased level of WNK includes, but is not limited to, hypertension and PHA II.

Therefore, the invention includes not only methods of inhibiting WNK expression, translation, and/or activity, but it also includes methods relating to increasing WNK expression, protein level, and/or activity since both decreasing and increasing WNK expression and/or activity can be useful in providing effective therapeutics.

Selection of any particular plasmid vector or other DNA vector is not a limiting factor in this invention and a wide variety of vectors is well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The invention thus includes a vector comprising an isolated nucleic acid encoding a mammalian WNK. The incorporation of a desired nucleic acid into a vector and the choice of vectors is well-known in the art as described in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The invention also includes cells, viruses, proviruses, and the like, containing such vectors. Methods for producing cells comprising vectors and/or exogenous nucleic acids are well-known in the art, and is detailed in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The nucleic acids encoding WNK may be cloned into various plasmid vectors. However, the present invention should not be construed to be limited to plasmids or to any particular vector. Instead, the present invention should be construed to encompass a wide plethora of vectors which are readily available and/or well-known in the art.

IV. Recombinant Cell

The invention includes a recombinant cell comprising, inter alia, an isolated nucleic acid encoding WNK, an antisense nucleic acid complementary thereto, a nucleic acid encoding an antibody that specifically binds WNK, and the like. In one aspect, the recombinant cell can be transiently transfected with a plasmid encoding a portion of the nucleic acid encoding WNK. The nucleic acid need not be integrated into the cell genome nor does it need to be expressed in the cell. Moreover, the cell may be a prokaryotic or a eukaryotic cell and the invention should not be construed to be limited to any particular cell line or cell type. Such cells include, but are not limited to, kidney cells, and the like. The invention should be construed to include any cell type into which a nucleic acid encoding a mammalian WNK (a transgene) is introduced, including, without limitation, a prokaryotic cell and a eukaryotic cell comprising an isolated nucleic acid encoding mammalian WNK.

When the cell is a eukaryotic cell, the cell may be any eukaryotic cell which, when the transgene of the invention is introduced therein, and the protein encoded by the desired gene is no longer expressed therefrom, a benefit is obtained. Such a benefit may include the fact that there has been provided a system in which lack of expression of the desired gene can be studied in vitro in the laboratory or in a mammal in which the cell resides, a system wherein cells comprising the introduced gene deletion can be used as research, diagnostic and therapeutic tools, and a system wherein animal models are generated which are useful for the development of new diagnostic and therapeutic tools for selected disease states in a mammal including, for example, hypertension, PHA II, renal diseases or disorders, salt readsorption, hyperkalemia, hydrogen ion secretion, and the like.

Alternatively, the invention includes a eukaryotic cell which, when the transgene of the invention is introduced therein, and the protein encoded by the desired gene is expressed therefrom where it was not previously present or expressed in the cell or where it is now expressed at a level or under circumstances different than that before the transgene was introduced, a benefit is obtained. Such a benefit may include the fact that there has been provided a system in the expression of the desired gene can be studied in vitro in the laboratory or in a mammal in which the cell resides, a system wherein cells comprising the introduced gene can be used as research, diagnostic and therapeutic tools, and a system wherein animal models are generated which are useful for the development of new diagnostic and therapeutic tools for selected disease states in a mammal.

Such cell expressing an isolated nucleic acid encoding WNK can be used to provide WNK to a cell, tissue, or whole animal where a higher level of WNK can be useful to treat or alleviate a disease, disorder or condition associated with low level of WNK expression and/or activity. Such diseases, disorders or conditions can include, but are not limited to, hypertension, PHA II, improper renal electrolyte handling, hyperkalemia, renal disease and disorders, and the like. Therefore, the invention includes a cell expressing WNK to increase or induce WNK expression, translation, and/or activity, where increasing WNK expression, protein level, and/or activity can be useful to treat or alleviate a disease, disorder or condition.

One of ordinary skill would appreciate, based upon the disclosure provided herein, that a “knock-in” or “knock-out” vector of the invention comprises at least two sequences homologous to two portions of the nucleic acid which is to be replaced or deleted, respectively. The two sequences are homologous with sequences that flank the gene; that is, one sequence is homologous with a region at or near the 5′ portion of the coding sequence of the nucleic acid encoding WNK and the other sequence is further downstream from the first. One skilled in the art would appreciate, based upon the disclosure provided herein, that the present invention is not limited to any specific flanking nucleic acid sequences. Instead, the targeting vector may comprise two sequences which remove some or all (i.e., a “knock-out” vector) or which insert (i.e., a “knock-in” vector) a nucleic acid encoding WNK, or a fragment thereof, from or into a mammalian genome, respectively. The crucial feature of the targeting vector is that it comprise sufficient portions of two sequences located towards opposite, i.e., 5′ and 3′, ends of the WNK open reading frames (ORF) in the case of a “knock-out” vector, to allow deletion/insertion by homologous recombination to occur such that all or a portion of the nucleic acid encoding WNK is deleted from or inserted into a location on a mammalian chromosome.

The design of transgenes and knock-in and knock-out targeting vectors is well-known in the art and is described in standard treatises such as Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York), and the like. The upstream and downstream portions flanking or within the WNK coding region to be used in the targeting vector may be easily selected based upon known methods and following the teachings disclosed herein based on the disclosure provided herein including the nucleic and amino acid sequences of both rat and human WNK. Armed with these sequences, one of ordinary skill in the art would be able to construct the transgenes and knock-out vectors of the invention.

The invention further includes a knock-out targeting vector comprising a nucleic acid encoding a selectable marker such as, for example, a nucleic acid encoding the neo^{R} gene thereby allowing the selection of transgenic a cell where the nucleic acid encoding WNK, or a portion thereof, has been deleted and replaced with the neomycin resistance gene by the cell's ability to grow in the presence of G418. However, the present invention should not be construed to be limited to neomycin resistance as a selectable marker. Rather, other selectable markers well-known in the art may be used in the knock-out targeting vector to allow selection of recombinant cells where the WNK gene has been deleted and/or inactivated and replaced by the nucleic acid encoding the selectable marker of choice. Methods of selecting and incorporating a selectable marker into a vector are well-known in the art and are described in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

One skilled in the art would appreciate, based upon this disclosure, that cells comprising decreased levels of WNK protein, decreased level of WNK activity, or both, include, but are not limited to, cells expressing inhibitors of WNK expression (e.g., antisense or ribozyme molecules).

Methods and compositions useful for maintaining mammalian cells in culture are well known in the art, wherein the mammalian cells are obtained from a mammal including, but not limited to, a rat and a human.

The recombinant cell of the invention can be used to study the effect of qualitative and quantitative alterations in WNK levels on renal electrolyte homeostasis systems. This is because the fact that WNK localizes to the subcellular regions of renal cells and tissues, and to the intracellular and tight junctions of renal cells. Further, the recombinant cell can be used to produce WNK for use for therapeutic and/or diagnostic purposes. That is, a recombinant cell expressing WNK can be used to produce large amounts of purified and isolated WNK that can be administered to treat or alleviate a disease, disorder or condition associated with or caused by an increased or inappropriate level of WNK.

Alternatively, recombinant cells expressing WNK can be administered in ex vivo and in vivo therapies where administering the recombinant cells thereby administers the protein to a cell, a tissue, and/or an animal. Additionally, the recombinant cells are useful for the discovery of WNK receptor(s) and WNK signaling and electrolyte handling pathways.

The recombinant cell of the invention may be used to study the effects of elevated or decreased WNK levels on cell homeostasis and electrolyte handling since WNK has been hypothesized to play a role in hypertension, PHA II, renal salt reabsorption, hyperkalemia, reduced renal hydrogen ion secretion, and the like.

The recombinant cell of the invention, wherein the cell has been engineered such that it does not express WNK, or expresses reduced or altered WNK lacking biological activity, can also be used in ex vivo and in vivo cell therapies where either an animal's own cells (e.g., kidney cells, and the like) or those of a syngeneic matched donor are recombinantly engineered as described elsewhere herein (e.g., by insertion of an antisense nucleic acid or a knock-out vector such that WNK expression and/or protein levels are thereby reduced in the recombinant cell), and the recombinant cell is administered to the recipient animal. In this way, recombinant cells that express WNK at a reduced level can be administered to an animal whose own cells express increased levels of WNK thereby treating or alleviating a disease, disorder or condition associated with or mediated by increased WNK expression as disclosed elsewhere herein.

V. Antibodies

The invention also includes an antibody that specifically binds WNK, or a fragment thereof.

One skilled in the art would understand, based upon the disclosure provided herein, that an antibody that specifically binds WNK, binds with a protein of the invention, such as, but not limited to human WNK1, human WNK2, human WNK3, human WNK4, rat WNK1, rat WNK2, rat WNK3, and rat WNK4, or an immunogenic portion thereof. In one embodiment, the antibody is directed to: human WNK1, comprising the amino acid sequence of SEQ ID NO:2, and human WNK4, comprising the amino acid sequence SEQ ID NO:4.

Polyclonal antibodies are generated by immunizing rabbits according to standard immunological techniques well-known in the art (see, e.g., Harlow et al., 1988, In: Antibodies, A Laboratory Manual, Cold Spring Harbor, N.Y.; and Wilson et al., 2001, Science 293: 1107-1112). Such,techniques include immunizing an animal with a chimeric protein comprising a portion of another protein such as a maltose binding protein or glutathione (GSH) tag polypeptide portion, and/or a moiety such that the WNK portion is rendered immunogenic (e.g., WNK conjugated with keyhole limpet hemocyanin, KLH) and a portion comprising the respective rodent and/or human WNK amino acid residues. The chimeric proteins are produced by cloning the appropriate nucleic acids encoding WNK (e.g:, SEQ ID NO:1 and SEQ ID NO:2) into a plasmid vector suitable for this purpose, such as but not limited to, pMAL-2 or pCMX.

However, the invention should not be construed as being limited solely to these antibodies or to these portions of the protein antigens. Rather, the invention should be construed to include other antibodies, as that term is defined elsewhere herein, to rat and human WNK, or portions thereof. Further, the present invention should be construed to encompass antibodies, inter alia, bind to WNK and they are able to bind WNK present on Western blots, in immunohistochemical staining of tissues thereby localizing WNK in the tissues, and in immunofluorescence microscopy of a cell transiently transfected with a nucleic acid encoding at least a portion of WNK.

One skilled in the art would appreciate, based upon the disclosure provided herein, that the antibody can specifically bind with any portion of the protein and the full-length protein can be used to generate antibodies specific therefor. However, the present invention is not limited to using the full-length protein as an immunogen. Rather, the present invention includes using an immunogenic portion of the protein to produce an antibody that specifically binds with mammalian WNK. That is, the invention includes immunizing an animal using an immunogenic portion, or antigenic determinant, of the WNK protein.

The antibodies can be produced by immunizing an animal such as, but not limited to, a rabbit or a mouse, with a protein of the invention, or a portion thereof, or by immunizing an animal using a protein comprising at least a portion of WNK, or a fusion protein including a tag polypeptide portion comprising, for example, a maltose binding protein tag polypeptide portion, covalently linked with a portion comprising the appropriate WNK amino acid residues. One skilled in the art would appreciate, based upon the disclosure provided herein, that smaller fragments of these proteins can also be used to produce antibodies that specifically bind WNK.

One skilled in the art would appreciate, based upon the disclosure provided herein, that various portions of an isolated WNK polypeptide can be used to generate antibodies to either highly conserved regions of WNK or to non-conserved regions of the polypeptide. As disclosed elsewhere herein, WNK comprises various conserved domains including, but not limited to, a section distal to the first and second putative coil domains from about amino acid residue 562 to about amino acid residue 565 and at about amino acid residue 1185 (in WNK4).

Once armed with the sequence of WNK and the detailed analysis localizing the various conserved and non-conserved domains of the protein, the skilled artisan would understand, based upon the disclosure provided herein, how to obtain antibodies specific for the various portions of a mammalian WNK polypeptide using methods well-known in the art or to be developed.

Further, the skilled artisan, based upon the disclosure provided herein, would appreciate that the non-conserved regions of a protein of interest can be more immunogenic than the highly conserved regions which are conserved among various organisms. Further, immunization using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of each WNK molecule can be used to produce antibodies that are specific only for that WNK and do not cross-react non-specifically with other WNKs or with other proteins.

Alternatively, the skilled artisan would also understand, based upon the disclosure provided herein, that antibodies developed using a region that is conserved among one or more WNK molecules can be used to produce antibodies that react specifically with one or more WNK molecule(s). Methods for producing antibodies that specifically bind with a conserved protein domain which may otherwise be less immunogenic than other portions of the protein are well-known in the art and include, but are not limited to, conjugating the protein fragment of interest to a molecule (e.g., keyhole limpet hemocyanin, and the like), thereby rendering the protein domain immunogenic, or by the use of adjuvants (e.g., Freund's complete and/or incomplete adjuvant, and the like), or both. Thus, the invention encompasses antibodies that recognize at least one WNK and antibodies that specifically bind with more than one WNK, including antibodies that specifically bind with all WNKs.

Indeed, the data disclosed herein demonstrate that antibodies have been produced using various portions of human WNK peptides. That is, as exemplified elsewhere herein, antibodies were produced in rabbits immunized with the peptide SQPGGSLAQAPTTSSQQ (SEQ ID NO:5) to produce anti-WNK1 antibodies and with the peptide MGQMRRPPGRNLRR (SEQ ID NO:6) to produce anti-WNK4 antibodies. The peptides were coupled to keyhole limpet hemocyamin. The skilled artisan would appreciate, based upon the disclosure provided herein, that portions of the WNK polypeptides of the invention can be used to generate antibodies of interest, and that the invention is not limited in any way to these peptides, or to any other fragments of WNK, but encompasses a wide plethora of peptides derived using the amino acid sequences for human WNK1 and WNK4 as disclosed herein.

One skilled in the art would appreciate, based upon the disclosure provided herein, which portions of WNK are less homologous with other proteins sharing conserved domains. However, the present invention is not limited to any particular domain; instead, the skilled artisan would understand that other non-conserved regions of the WNK proteins of the invention can be used to produce the antibodies of the invention as disclosed herein.

Therefore, the skilled artisan would appreciate, based upon the disclosure provided herein, that the present invention encompasses antibodies that neutralize and/or inhibit WNK activity (e.g., by inhibiting necessary WNK receptor/ligand interactions) which antibodies can recognize one or more WNKs, including, but not limited to, rat WNKs and human WNKs, as well as WNKs from various species (e.g., mouse, non-human primates).

One skilled in the art would also understand, based upon the disclosure provided herein, that it may be advantageous to inhibit the activity and/or expression of one type of WNK molecule without affecting the activity and/or expression of other WNK molecules. For example, it may be beneficial to inhibit WNK4 expression to treat hypertension where WNK4 is over-expressed or exhibits increased activity in kidney cells, while not inhibiting the expression and/or activity of WNK1 in other tissues where the existing level of WNK1 in the these other tissues is necessary for continued proper functioning of cellular processes in that tissue. Thus, whether inhibition of WNK expression and/or activity is achieved using antibodies, antisense nucleic acids, and the like, one skilled in the art would appreciate, based upon the disclosure provided herein, that the present invention encompasses selectively affecting one or more WNK molecules and, in certain cases, the invention encompasses inhibiting the expression or activity of all WNKs. Whether one or more WNKs should be affected can be readily determined by the skilled artisan based on which disease, disorder or condition is being treated, and the specific tissue (e.g., kidneys) being targeted.

The invention should not be construed as being limited solely to the antibodies disclosed herein or to any particular immunogenic portion of the proteins of the invention. Rather, the invention should be construed to include other antibodies, as that term is defined elsewhere herein, to WNK, or portions thereof, or to proteins sharing greater than 86% homology with a polypeptide having the amino acid sequence of at least one of SEQ ID NO:2 and SEQ ID NO:4. Preferably, the polypeptide is about 87% homologous, more preferably, about 90% homologous, even more preferably, about 95% homologous, and most preferably, about 99% homologous to at least one of human WNK1 (SEQ ID NO:2) and human WNK2 (SEQ ID NO:4). More preferably, the polypeptide that specifically binds with an antibody specific for mammalian WNK is at least one of human WNK1 and human WNK4. Most preferably, the polypeptide that specifically binds with an antibody that specifically binds with a mammalian WNK is at least one of SEQ ID NO: 2 and SEQ ID NO:4.

The invention encompasses polyclonal, monoclonal, synthetic antibodies, and the like. One skilled in the art would understand, based upon the disclosure provided herein, that the crucial feature of the antibody of the invention is that the antibody bind specifically with WNK. That is, the antibody of the invention recognizes WNK, or a fragment thereof (e.g., an immunogenic portion or antigenic determinant thereof), on Western blots, in immunostaining of cells, and immunoprecipitates WNK using standard methods well-known in the art.

One skilled in the art would appreciate, based upon the disclosure provided herein, that the antibodies can be used to localize the relevant protein in a cell and to study the role(s) of the antigen recognized thereby in cell processes. Moreover, the antibodies can be used to detect and or measure the amount of protein present in a biological sample using well-known methods such as, but not limited to, Western blotting and enzyme-linked immunosorbent assay (ELISA). Moreover, the antibodies can be used to immunoprecipitate and/or immuno-affinity purify their cognate antigen using methods well-known in the art. In addition, the antibody can be used to decrease the level of WNK in a cell thereby inhibiting the effect(s) of WNK in a cell. Thus, by administering the antibody to a cell or to the tissues of an animal or to the animal itself, the required WNK receptor/ligand interactions are therefore inhibited such that the effect of WNK mediated activity are also inhibited. One skilled in the art would understand, based upon the disclosure provided herein, that detectable effects upon inhibiting WNK ligand/receptor interaction and/or activity using an anti-WNK antibody can include, but are not limited to, decreased hypertension, improved renal electrolyte handling, reduced renal salt reabsorption, reduced hyperkalemia, reduced symptoms of PHA II, and the like.

One skilled in the art would appreciate, based upon the disclosure provided herein, that the invention encompasses administering an antibody that specifically binds with WNK orally, parenterally, or both, to inhibit WNK function in the renal system.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein.

Further, the antibody of the invention may be “humanized” using the technology described in, for example, Wright et al. (1992, Critical Rev. Immunol. 12:125-168), and in the references cited therein, and in Gu et al. (1997, Thrombosis and Hematocyst 77:755-759), and other methods of humanizing antibodies well-known in the art or to be developed.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., (1989, Molecular Cloning: A Laboratory Manual, Cold Spring Harbor Laboratory, New York).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (1992, Critical Rev. Immunol. 12:125-168).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

In addition to administering an antibody to a cell to inhibit the activity and/or expression of mammalian WNK, the invention encompasses administering an antibody that specifically binds with a mammalian WNK, or a nucleic acid encoding the antibody, wherein the molecule further comprises an intracellular retention sequence such that antibody binds with the WNK and prevents its expression at the cell surface, or at other locations throughout the subcellular milieu. Such antibodies, frequently referred to as “intrabodies”, are well known in the art and are described in, for example, Marasco et al. (U.S. Pat. No. 6,004,490) and Beerli et al. (1996, Breast Cancer Research and Treatment 38:11-17). Thus, the invention encompasses methods comprising inhibiting binding of WNK with a receptor of interest where the receptor is present on the cell surface (e.g., antibodies, chemical compounds, small molecules, peptidomimetics, drugs, and the like), as well as methods of inhibiting the binding comprising inhibiting the receptor being present on the cell surface (e.g., ribozymes, antisense molecules, intrabodies, and the like), and such methods as become known in the future for inhibiting ligand:receptor interaction on the cell surface between WNK and the WNK receptor.

VI. Methods and Compositions

A. Methods and Compositions for Treating Diseases, Disorders, and Conditions

In one aspect of the present invention, there is provided a method to treat diseases, disorders, and conditions associated with or mediated mammalian WNK. Such disease, disorders and conditions include, but are not limited to hypertension, PHA II, hyperkalemia, renal electrolyte handling disorders, and the like. In the present embodiment, hypertension is treated by administering to a mammal a WNK inhibitor. In a preferred embodiment of the present invention, the WNK inhibitor is not a Rho kinase inhibitor. WNK inhibitors are well known to those of ordinary skill in the art, and may include, but are not limited to bisindolylmaleimide I, H-89 dihydrochloride, KN-93, ML-7, protein kinase G inhibitor, staurosporine, H-7, and KT-5926.

The method comprises administering to a human a WNK expression inhibiting amount of a WNK inhibitor. This is because, the data disclosed herein demonstrate that WNK is associated with hypertension. That is, the data demonstrate that increased expression of WNK, (e.g., WNK1), is correlated with hypertension, e.g., PHA II. Further, the data disclosed herein demonstrate that expression of a mutant form of WNK4 is also correlated with hypertension. More specifically, WNK4 mutations comprising a substitution of Gln⁵⁶⁵ for Glu, a substitution of Asp⁵⁶⁴ for Ala, a substitution of Glu⁵⁶² for Lys, and a substitution of Arg¹¹⁸⁵ for Cys are correlated with, among other things, hypertension, PHA II, hyperkalemia, increased renal salt reabsorption, metabolic acidosis, and an overall defect in renal electrolyte handling.

Therefore, decreasing expression of WNK (e.g., WNK1, WNK2, WNK3, and WNK4), or decreasing expression and/or activity of mutant forms of WNK with, for example, a chemical compound, a peptidomimetic, a small molecule, ribozymes, antisense nucleic acids, antibodies, and intrabodies that inhibit WNK and/or mutant WNK, provides a method of treating hypertension, and as disclosed herein, PHA II. Thus, one of ordinary skill in the art would understand that inhibiting WNK, which can be accomplished by a variety of methods as more fully set forth elsewhere herein, is a useful treatment for hypertension, and PHA II.

The compositions of the present invention can be used to administer WNK to a cell, a tissue, or an animal or to inhibit expression of WNK in a cell, a tissue, or an animal. The compositions are useful to treat a disease, disorder or condition mediated by altered expression of WNK such that decreasing or increasing WNK expression or the level of the protein in a cell, tissue, or animal, is beneficial to the animal. That is, where a disease, disorder or condition in an animal is mediated by or associate with altered level of WNK expression or protein level, the composition can be used to modulate such expression or protein level of WNK.

In one embodiment of the present invention, the inhibitors of mammalian WNK gene expression may be administered singly or in any combination thereof Further, inhibitors of WNK may be administered singly or in any combination thereof in a temporal sense, in that they may be administered simultaneously, before, and/or after each other. One of ordinary skill in the art will appreciate the use of WNK inhibitors or inhibitors of WNK gene expression to treat hypertension and will use the inhibitors detailed herein alone or in any combination to effect such results.

The invention also encompasses the use of pharmaceutical compositions comprising an appropriate WNK inhibitor to practice the methods of the invention, the compositions comprising an appropriate WNK inhibitor and a pharmaceutically-acceptable carrier.

As used herein, the term “pharmaceutically-acceptable carrier” means a chemical composition with which an appropriate WNK inhibitor may be combined and which, following the combination, can be used to administer the appropriate WNK inhibitor to a mammal.

The pharmaceutical compositions useful for practicing the invention may be administered to deliver a dose of between 1 ng/kg/day and 100 mg/kg/day.

Pharmaceutical compositions that are useful in the methods of the invention may be administered systemically in oral solid formulations, ophthalmic, suppository, aerosol, topical or other similar formulations. In addition to the appropriate WNK kinase inhibitor, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate WNK kinase inhibitor according to the methods of the invention.

Compounds which are identified using any of the methods described herein may be formulated and administered to a mammal for treatment of the diseases disclosed herein are now described.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats and dogs, and birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

B. Methods of Identifying Compounds

The present invention further includes a method of identifying a compound that affects expression of mammalian WNK, including, but not limited to hWNK1 and hWNK4, in a cell. The method comprises contacting a cell with a test compound and comparing the level of expression of WNK in the cell so contacted with the level of expression of WNK in an otherwise identical cell not contacted with the compound. If the level of expression of WNK is higher or lower in the cell contacted with the test compound compared to the level of expression of WNK in the otherwise identical cell not contacted with the test compound, this is an indication that the test compound affects expression of WNK in a cell.

The invention encompasses methods to identify a compound that affects expression of WNK. One skilled in the art would appreciate, based upon the disclosure provided herein, that assessing the level of WNK, or both, can be performed using probes (e.g., antibodies and/or nucleic acid probes that specifically bind with of WNK), or other methods disclosed herein, such that the method can identify a compound that selectively affects expression of WNK. Such compounds are useful for inhibiting expression of WNK. One skilled in the art would understand that such compounds can be useful for inhibiting a disease, disorder, or condition mediated by and/or associated with increased expression of WNK, e.g., increased levels of WNK is associated with, among other things, hypertension and PHA II. Thus, the skilled artisan would appreciate, based on the disclosure provided herein, that it may useful to decrease expression of WNK.

Similarly, the present invention includes a method of identifying a compound that reduces or inhibits expression of WNK in a cell. The method comprises contacting a cell with a test compound and comparing the level of expression of WNK in the cell contacted with the compound with the level of expression of WNK in an otherwise identical cell, which is not contacted with the compound. If the level of expression of WNK is lower in the cell contacted with the compound compared to the level in the cell that was not contacted with the compound, then that is an indication that the test compound reduces expression of WNK in a cell.

A compound that inhibits WNK expression in a cell is useful since it has been demonstrated herein that gain of function mutations in mammalian WNK are associated with hypertension, PHA II, and reduced electrolyte handling. Thus, methods of identifying a compound that inhibits WNK expression can be used to identify useful compounds to treat various diseases, including, but not limited to, hypertension and PHA II. Such a compound is included in the present invention.

One skilled in the art would appreciate, based on the disclosure provided herein, that the level of expression of WNK in the cell may be measured by determining the level of expression of mRNA encoding WNK. Alternatively, the level of expression of WNK can be determined by using immunological methods to assess WNK production. Further, nucleic acid-based detection methods, such as Northern blot and PCR assays and the like, can be used as well. In addition, the level of WNK activity and/or expression in a cell can also be assessed by determining the level of various parameters which can be affected by WNK activity and/or expression, such as, for example, renal electrolyte handling, salt reabsorption, potassium levels, and the like. Thus, one skilled in the art would appreciate, based upon the disclosure and reduction to practice provided herein, that there are a multitude of methods that are well-known in the art which can be used to asses the level of WNK expression in a cell, including those disclosed herein and others which may be developed in the future.

VI. Diagnostics

The present invention encompasses methods for the diagnosis of a disease, disorder, or condition related to altered expression of WNK. The skilled artisan will appreciate, when armed with the present disclosure and the data disclosed herein, that the altered expression of WNK is associated with or mediates a variety of diseases relating to a defect in renal electrolyte handling, including PHA II, hypertension, hyperkalemia, increased salt reabsorption, and the like.

The method comprises detecting the level of WNK expression in a first human and comparing the level of WNK expression in the first human to the level of WNK expression in a second human, the second human not having a disease associated with or mediated by altered expression of WNK, thereby identifying a human afflicted with a disease associate with altered WNK expression. One of skill in the art, in light of the present disclosure, will readily understand that altered expression of WNK is closely associated with a gain of function phenotype, leading to the diseases and symptoms discussed herein.

The present invention further includes a method for detecting a mutation in a WNK allele in a human. As demonstrated by the data disclosed herein, mutations in WNK alleles are associated with, among other things, PHA II, hypertension, and hyperkalemia. As detailed more specifically elsewhere herein, specific mutations in the conserved regions of WNK4 and in an intron of WNK1 result in dysregulation of renal electrolyte balance. Therefore, a method of detecting any mutation in a WNK allele that effects WNK such that it is correlated with hypertension can be a powerful and novel diagnostic tool for detecting such diseases.

In one aspect, the method comprises comparing the nucleic acid sequence encoding WNK of a human suspected of having a mutation in WNK with the nucleic acid sequence of a normal human without a mutation in WNK. Any differences in the nucleic acid sequences encoding WNK between the two humans serves as a method of detection for mutations in a WNK allele in a human.

In another aspect, the method includes detecting a mutation in a WNK allele of a human by comparing the genomic nucleic acid sequence encoding WNK of a human thought to have a mutation in WNK with that of a normal human that does not have a mutation in the genomic nucleic acid sequence encoding WNK. Thereby, any difference between the allele of the human thought to have a mutation in WNK and the allele of the human without a mutation is indicative of a mutation in the genomic nucleic acid sequence encoding WNK.

The routineer will further appreciate that the methods disclosed elsewhere herein will enable one of skill in the art to detect altered WNK expression and mutations in a WNK allele in a variety of cells and tissues, including but not limited to, blood, renal biopsies, and the like. Methods which are well known in the art, and are further exemplified herein, include PCR, RT-PCR, Northern blotting, Southern blotting, Western blotting, single strand conformational polymorphism, and other methods known or to be discovered.

The present invention also includes to detect mutations in a nucleic acid of the invention in order to determine if a human has a mutated gene since such a human is potentially at risk for a disease, disorder, or condition associated with altered expression or activity of WNK. This is because it is well-known that most mutations are deleterious such that a human having a mutation in the nucleic acid encoding WNK is more likely than not to be negatively impacted by such mutation which is potentially associated with altered expression or activity of the protein encoded by the mutated nucleic acid.

In certain embodiments, the methods include detecting, in a sample of cells obtained from the human, the presence or absence of a mutation characterized by at least one of an alteration of a nucleic acid encoding a WNK, or the altered expression of the gene encoding WNK. For example, such mutations can be detected by ascertaining the existence of at least one of: 1) a deletion of one or more nucleotides from the nucleic acid encoding or introns comprising the genomic DNA of WNK1; 2) a substitution of one or more nucleotides of the nucleic acid encoding WNK; 3) an alteration in the level of a messenger RNA transcript of the nucleic acid encoding WNK4) an aberrant modification of the gene, such as of the methylation pattern of the genomic DNA; 5) a non-wild type splicing pattern of a messenger RNA transcript of the nucleic acid encoding WNK; 6) a non-wild type level of the protein encoded by the nucleic acid encoding WNK. As described herein, there are a large number of assay techniques known in the art which can be used for detecting such mutations in a nucleic acid encoding a known protein. Thus, once armed with the teachings set forth herein, including the nucleic and amino acid sequences of human and rat WNK one skilled in the art would be able to detect a mutation in the WNK gene of genomic DNA.

In certain embodiments, detection of the mutation involves the use of an primer in a polymerase chain reaction (PCR; see, e.g., U.S. Pat. Nos. 4,683,195 and 4,683,202), such as anchor PCR or RACE PCR, or, alternatively, in a ligation chain reaction (LCR; see, e.g., Landegran et al., 1988, Science 241:1077-1080; and Nakazawa et al., 1994, Proc. Natl. Acad. Sci. USA 91:360-364), the latter of which can be particularly useful for detecting point mutations in a gene (see, e.g., Abravaya et al., 1995, Nucleic Acids Res. 23:675-682). This method can include the steps of collecting a sample of cells from a patient, isolating nucleic acid (e.g., genomic, mRNA, or both) from the cells of the sample, contacting the nucleic acid sample with one or more primers which specifically hybridize with the selected gene under conditions such that hybridization and amplification of the gene (if present) occurs, and detecting the presence or absence of an amplification product. The method can also include detecting the size of the amplification product and comparing the length to the length of a corresponding product obtained in the same manner from a control sample. PCR, LCR, or both can be used as a preliminary amplification step in conjunction with any of the techniques used for detecting mutations described herein.

Alternative amplification methods include: self-sustained sequence replication (Guatelli et al., 1990, Proc. Natl. Acad. Sci. USA 87:1874-1878), transcriptional amplification system (Kwoh, et al., 1989, Proc. Natl. Acad. Sci. USA 86:1173-1177), Q-Beta Replicase (Lizardi et al., 1988, Bio/Technology 6:1197), or any other nucleic acid amplification method, followed by the detection of the amplified molecules using any of a variety of techniques well known to those of skill in the art. These detection schemes are especially useful for detection of nucleic acid molecules if such molecules are present in very low numbers.

In an alternative embodiment, mutations in a selected gene can be identified in a sample by detecting alterations in restriction enzyme cleavage patterns. For example, sample and control DNA is isolated, (optionally) amplified, digested with one or more restriction endonucleases, and fragment length sizes are determined by gel electrophoresis and compared. Differences in fragment length sizes between sample and control DNA (i.e., restriction fragment length polymorphism, RFLP) indicates occurrence of mutations or other sequence differences in the sample DNA compared with control, wild type DNA.

Moreover, sequence specific ribozymes (see, e.g., U.S. Pat. No. 5,498,531) can be used to detect the presence of specific mutations by development or loss of a ribozyme cleavage site.

In other embodiments, genetic mutations are identified by hybridizing a sample and control nucleic acids, e.g., DNA or RNA, with high density arrays containing hundreds or thousands of oligonucleotides probes (Cronin et al. (1996) Human Mutation 7:244-255; Kozal et al. (1996) Nature Medicine 2:753-759.

In addition, any of a variety of sequencing methods known in the art can be used to directly sequence the selected gene and detect mutations by comparing the sequence of the sample nucleic acids with the corresponding wild-type (control) sequence (see, e.g., Maxam and Gilbert, 1977, Proc. Natl. Acad. Sci. USA 74:560; Sanger, 1977, Proc. Natl. Acad. Sci. USA 74:5463. It is also contemplated that any of a variety of automated sequencing procedures can be used when performing the diagnostic assays (as reviewed in 1995, Bio/Techniques 19:448). Such automated sequencing methods include mass spectrometry (see, e.g., PCT Publication No. WO 94/16101; Cohen et al., 1996, Adv. Chromatogr. 36:127-162; Griffin et al., 1993, Appl. Biochem. Biotechnol. 38:147-159).

Other methods for detecting mutations in a selected gene include methods involving protection from cleavage agents to detect mismatched bases in RNA/RNA or RNA/DNA heteroduplexes as described in, e.g., Myers et al. (1985, Science 230:1242). In essence, hybridizing RNA or DNA containing wild-type sequence with potentially mutant RNA or DNA obtained from a tissue sample and subsequent treatment of the duplexes formed with an agent(s) (e.g., S1 nuclease, hydroxylamine or osmium tetroxide with piperidine, DNA mismatch enzymes such as mutY from E. coli or mammalian thymidine DNA glycosylase) that cleaves single-stranded regions of duplex detects base pair mismatches between the control and sample strands. Following digestion of the mismatched regions, the resulting material is separated by size on denaturing polyacrylamide gels to determine the site of the mutated or mismatched region (see, e.g., Cotton et al., 1988, Proc. Natl. Acad. Sci. USA 85:4397; Saleeba et al., 1992, Methods Enzymol. 217:286-295).

In other embodiments, alterations in electrophoretic mobility are used to identify mutations in genes. For example, single strand conformation polymorphism (SSCP) analysis can be used to detect differences in electrophoretic mobility between mutant and wild type nucleic acids as described in Orita et al. (1989, Proc. Natl. Acad. Sci. USA 86:2766), Cotton (993, Mutat. Res. 285:125-144), and Hayashi (1992, Genet. Anal. Tech. Appl. 9:73-79).

In yet another embodiment, the movement of mutant or wild-type fragments in polyacrylamide gels containing a gradient of denaturant is assayed using denaturing gradient gel electrophoresis (DGGE), as described (Myers et al., 1985, Nature 313:495).

Examples of other techniques for detecting point mutations include, but are not limited to, selective oligonucleotide hybridization, selective amplification, and selective primer extension (see, e.g., Saiki et al., 1986, Nature 324:163; Saiki et al., 1989, Proc. Natl. Acad. Sci. USA 86:6230).

Alternatively, allele specific amplification technology can be used in conjunction with the methods of the invention as described in, for example, Gibbs et al. (1989, Nucleic Acids Res. 17:2437-2448), Prossner (1993, Tibtech 11:238), Gasparini et al. (1992, Mol. Cell Probes 6:1), and Barany (1991, Proc. Natl. Acad. Sci. USA 88:189).

The methods described herein can be performed, for example, using pre-packaged diagnostic kits comprising at least one probe nucleic acid or antibody reagent described herein. Such kits can be used, for example, to diagnose a human patient exhibiting a disease, disorder, or condition involving a nucleic acid encoding WNK. Furthermore, any cell type or tissue in which the polypeptide of the invention is expressed, e.g., a blood sample containing peripheral blood leukocytes for proteins which are secreted or which occur on or in peripheral blood leukocytes, as well as cells and/or tissue from the kidney, heart, and skeletal muscle, can be used in the prognostic assays described herein.

VII. Kits

The present invention further encompasses various kits relating to detecting a nucleic acid encoding a mammalian WNK. This is because, as demonstrated by the data disclosed herein, WNK plays a role in the regulation of renal electrolyte balance, and therefore, dysregulation of WNK results in, among other things, hypertension, PHA II, hyperkalemia, increased renal salt reabsorption, altered pH homeostasis, and the like. Therefore, a kit for the detection of the nucleic acid encoding WNK, and for detecting mutations therein, is a novel and effective tool in both diagnosing and combating these diseases.

The kit comprises various nucleic acid molecules for the detection of a nucleic acid encoding a mammalian WNK, preferably a mutant mammalian WNK, and an instructional material detailing the use of the kit. The nucleic acid molecules can comprise a first nucleic acid having a sequence complementary with at least a portion of at least one of SEQ ID NO:1 and SEQ ID NO:3, and a second nucleic acid having the sequence complementary with at least a portion of at least one of SEQ ID NO:1 and SEQ ID NO:3, but downstream from the previous sequence, such that the sequences flank at least a portion of one of SEQ ID NO:1 and SEQ ID NO:3. The nucleic acid molecules of the kit specifically bind with a nucleic acid encoding a mammalian WNK, or a fragment thereof, thereby facilitating the detection of WNK in a human or biological sample. The kit further comprises an oligonucleotide primer or probe that is complementary to at least a portion of a nucleic acid molecule having a sequence of SEQ ID NO:1 and SEQ ID NO:3.

The skilled artisan will understand, when armed with the present disclosure and the data contained herein, that methods for using the kit, of which will be supplied in the instructional material, comprise methods well known in the art for detecting a nucleic acid molecule in an animal or a biological sample therefrom. Such methods include, but are not limited to, PCR, RT-PCR, Southern blotting, Northern blotting, and other methods known in the art or to be discovered.

### EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein. The examples included herein are described in Wilson et al. (2001, Science 293: 1107-1112), incorporated herein by reference in its entirety.

The materials and methods used in the experiments presented in these Examples are now described.

Identification of PHA II kindreds: The kindred (K22) comprised twenty-eight individuals in total, in which ten living members displayed typical features of PHA II, including hypertension (140/90 mmHg in adults), hyperkalemia (mean serum potassium level of 6.2 mM; normal is 3.5-5.0), normal glomerular filtration, suppressed plasma renin activity, normal or elevated aldosterone, hyperchloremia (mean serum chloride of 112 mM; normal is 95-105), and reduced serum bicarbonate levels (mean 17.5 mM, normal is 22-28). These clinical symptoms of PHA II were absent in unaffected members of the kindred. Other kindreds (K4, K11, K13, K21 and K23) were also selected for confirmatory and research purposes (Disse-Nicodème et al., 2000, Am. J. Hum. Genet., 67:302; Lee et al., 1979, Q. J. Med., 48:245; Lee et al., 1980, Lancet, 1:879; Farfel et al., 1978, Arch. Intern. Med., 138:1828; Baz et al., 1990, Presse Med., 19;1981).

Identification of WNK kinase loci and genome-wide linkage analysis: A genome scan was performed using 380 polymorphic markers spaced at 10 centimorgan (cM) intervals. Lod scores were calculated specifying PHA II as an autosomal dominant trait (disease allele frequency of 0.0001, penetrance of 99%, phenocopy rate of 0.0001) (Lathrop et al., 1984, Proc. Nat. Acad. Sci. U.S.A. 81:3443). Publicly available genomic sequence, cDNA sequences and ESTs from human, mouse and rat were used to predict genomic and mRNA structures of hWNK1 and hWNK4 according to methods known in the art. A comparison of the genomic sequence of the BAC clone RPCI1 1-388A16 with ESTs assembled from public databases reveals that exons 1-12 of hWNK1 are contained on the clone. The remaining 16 exons of hWNK1 are located on an overlapping BAC clone RP11-359B12 (GenBank Account No. AC004803). EST database searches, GENSCAN exon predictions, and PCR amplification from kidney cDNA provide no evidence for transcripts or exons within the deleted interval. Exons 9, 11 and 12 are contained in some, but not all transcripts, indicating alternative splicing. A BLAST search of the assembled hWNK1 cDNA sequence was preferred according to well-known methods (http://www.ncbi.nlm.nih.gov/BLAST) and the search identified a partially sequenced BAC clone, RP11-506G7 (GenBank Account No. AC016889), containing significant sequence similarity to the kinase domain of hWNK1. A comparison of ESTs with the BAC genomic sequence identified exons later found to comprise the 3′ end of the gene. ESTs, exon predictions by GENSCAN, and homology both with hWNK1 and with mouse genomic sequence containing the WNK4 ortholog (mouse BAC RP23-286N22; GenBank Account No. AC025424) were used to design primers for PCR amplification from human kidney cDNA. Amplified products were sequenced to confirm the intron-exon structure of the gene.

Identification of a deletion in the linked interval: Sequencing of the interval containing the DS12S94 locus permitted a BLAST search that revealed D12S342, D12SS94, and D12S91 are on the bacterial artificial chromosome (BAC) clone RPCI11-388A16 (GenBank Account No. AC004765). Three additional dinucleotide repeats were identified on the BAC and are designated STS42K, STS45K, and STS60K. All dinucleotide repeats are polymorphic with observed heterozygosities ranging from 0.30 to 0.60 in caucasians. Other deletions in the segment shared between the K22 and K4 deletions were sought in controls by quantitative PCR. Primers amplifying a product within this segment (nucleotides 62653-62856, GenBank Account No. AC004765) and a product of similar size from an unlinked gene (KCC4, GenBank Account No. AF105365) were used in the same reactions to direct PCR using DNA from individuals of K22, 40 unrelated unaffected subjects and affected members of other PHA II kindreds as template. The ratio of WNK1 amplification relative to control amplification was quantitated; the mean of the ratio for each subject was determined based on at least four (4) independent measurements. Individuals with heterozygous WNK1 deletions clearly demonstrated markedly lower ratios than their wild-type relatives, with no overlap between the two distributions. None of 40 unrelated subjects or members of other PHA II kindreds studied demonstrated ratios in the range of patients with deletions, indicating that deletions of this segment must be rare in the population. Southern blotting was performed by hybridizing 3 probes across this interval to the genomic DNA of 20 control individuals where the DNA was digested with enzyme EcoRV. No fragments other than those predicted by the wild-type genomic sequence were detected. A smaller deletion lying in the proximal portion of intron 1 and which does not overlap with PHA II deletions was also identified. This deletion removes 7983 nucleotides from intron 1 (nucleotides 27074-35056 in GenBank Account No. AC004765). The deletion endpoints lie in a 5 bp segment of sequence identity. The frequency of this allele was estimated at 10% by PCR genotyping of 70 unrelated unaffected subjects.

Determination of deletion endpoints by Southern blotting: Using the genomic sequence of the interval to define probes and restriction enzyme cleavage fragments, the endpoints of the deletion were determined by using Southern blotting. Genomic DNA (gDNA) was digested with PvuII and Southern blotting was performed using ³²P-labeled probe 1 (nucleotide 24593 to 25113) and probe 2 (nucleotides 78807 to 79341) from BAC clone RPCI1 1-388A16 (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

Identification of deletion by Polymerase Chain Reaction (PCR): PCR was used to amplify the product spanning the two identified deletion endpoints. A primer pair based on the genomic DNA that is normally separated by 42 kb in both unaffected members of the kindred and 160 unrelated controls was designed. Alternative primer pairs were used for confirmation. Another primer pair were designed to identify the deletion in the K4 kindred. PCR products were visualized by agarose gel electrophoresis and subjected to DNA sequencing.

Quantitative PCR: RNA was extracted from leukocytes of members of the K4 kindred and control subjects. RNA was purified by guanidinium thiocyanate-phenol-chloroform followed by treatment with RNAse free DNAse. First strand CDNA was synthesized followed by amplification with specific primers from exons 7 and 8 of hWNK1, GAPDH, and 18s ribosomal RNA locus. Products were quantitated using an ABI PRISM 7700 instrument (Applied Biosystems, Foster City, Calif.). The threshold cycle number (C_{T}) at which fluorescence reaches ten times the standard deviation of the baseline measured with no template was determined in triplicate. C_{T} is linearly related to the log of initial mRNA copy number. WNK1 level in each subject was evaluated as the WNK1:GAPDH ratio, 2^{-ΔCT}, where ΔCT is defined as the difference between the mean C_{T} value from WNK1 and GAPDH. Similar estimates of WNK1 transcript levels were obtained by comparison to 18S ribosomal RNA.

Preparation and characterization of anti-WNK1 and anti-WNK4 antibodies: Rabbits were immunized with the peptide SQPGGSLAQAPTTSSQQ (SEQ ID NO:5) to produce anti-WNK1 antibodies and with the peptide MGQMRRPPGRNLRR (SEQ ID NO:6) to produce anti-WNK4 antibodies. Both peptides were coupled to keyhole limpet hemocyamin (Sigma Chemical Company, St. Louis Mo.). Rabbits were immunized with successive injections and serum was harvested. Antibodies were affinity purified using the immunizing peptide linked to aniodoacetyl cross-linked agarose column (Harlow et al., 1988, Antibodies: A Laboratory Manual, Cold Spring Harbor, N.Y.).

cDNAs comprising the immunizing peptides were separately cloned into pGEX4T-1 (Pharmacia, Peapack N.J.) and transformed into E. coli to produce GST fusion proteins. Lysates expressing GST-WNK constructs were prepared by inducing log-phase cultures with 0.1 mM isopropyl β-D-1-thiogalactopyranoside (IPTG, Sigma Chemical Company, St. Louis Mo.). Bacteria were pelleted by centrifugation and resuspended in 2×sample buffer and boiled (Sambrook et al., 1989, Molecular Cloning: A Laboratory Manual, Cold Spring Harbor Laboratory, New York). Samples were subjected to 12% polyacrylamide gel electrophoresis (PAGE, Laemmli et al., 1970, Nature 227:680). Proteins were transferred to nitrocellulose membranes and immunoblotting was performed with anti-WNK sera and with anti-GST antibodies. Confirmatory immunoblotting was performed with Dounce homogenized whole kidney lysates prepared from 8-week-old mice (Kontes, Inc., Vineland, N.J.). Debris and nuclei were pelleted by centrifugation. The resulting samples were subjected to 5% PAGE, transferred to nitrocellulose membranes and immunoblotted with anti-WNK sera.

The results of the experiments presented in this Example are now described.

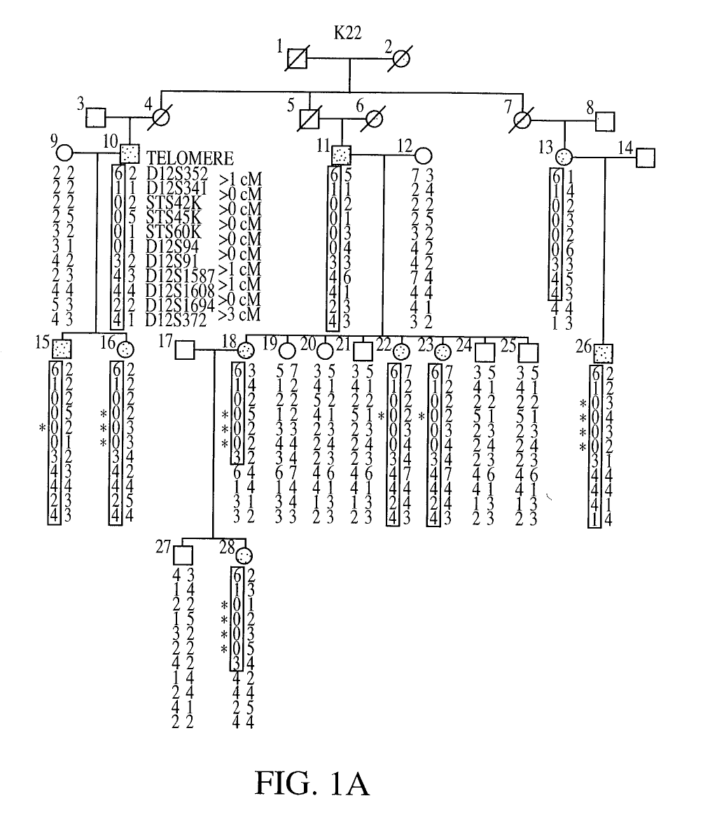

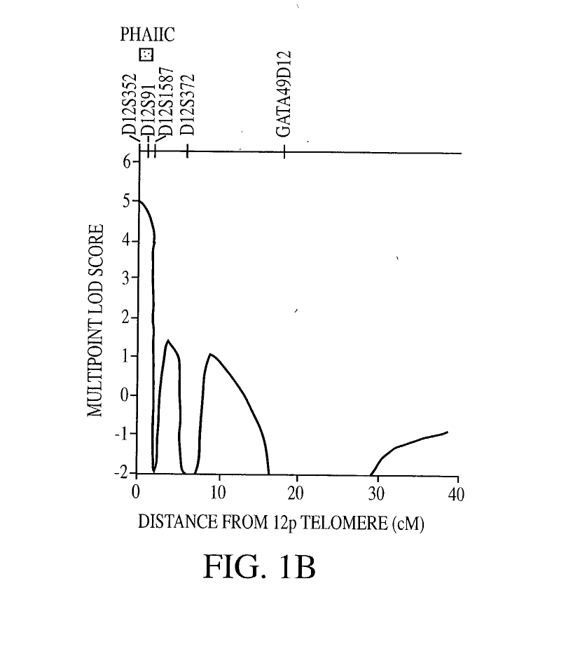

Linkage Analysis: An analysis of the K22 kindred indicated that the inheritance of the PHA II trait was consistent with autosomal dominant transmission with high penetrance (FIG. 1A). A genome-wide analysis of the linkage of PHA II indicated linkage to the most telomeric 2 cM segment of chromosome 12p, with a multipoint lod score of 5.07 (odds ratio greater than 10⁵:1 in favor of linkage) (FIG. 1B).

Identification of a deletion in the linked interval: Genotyping of additional loci in the completely linked telomeric interval revealed that one locus, D12S94, violated simple Mendelian transmission in that all affected members were homozygous at this locus, and in all four informative matings, affected offspring did not inherit the allele from their affected parent. This indicated a null allele consistent with a deletion in the disease linked chromosome segment.

The identification of three polymorphic loci close to D12S94 and subsequent genotyping of these loci in the K22 kindred indicated the presence of a null allele on the disease chromosome, with no evidence of transmission from affected parents to affected offspring. Flanking loci D12S341 and D12S91 demonstrated no violations of Mendelian inheritance indicating a deletion in the interval between D12S341 and D12S91.

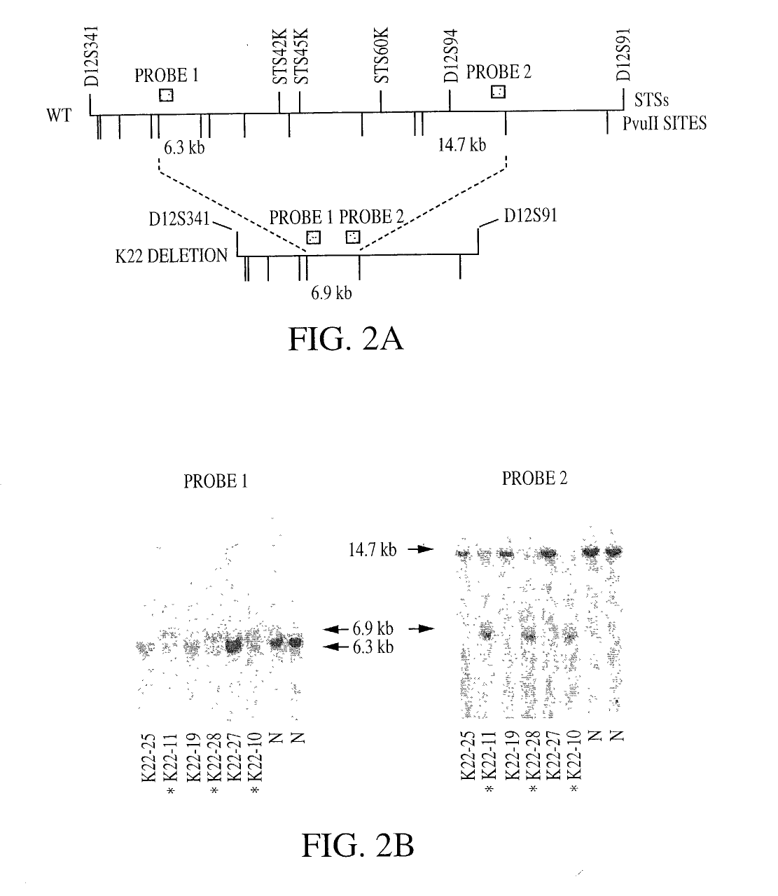

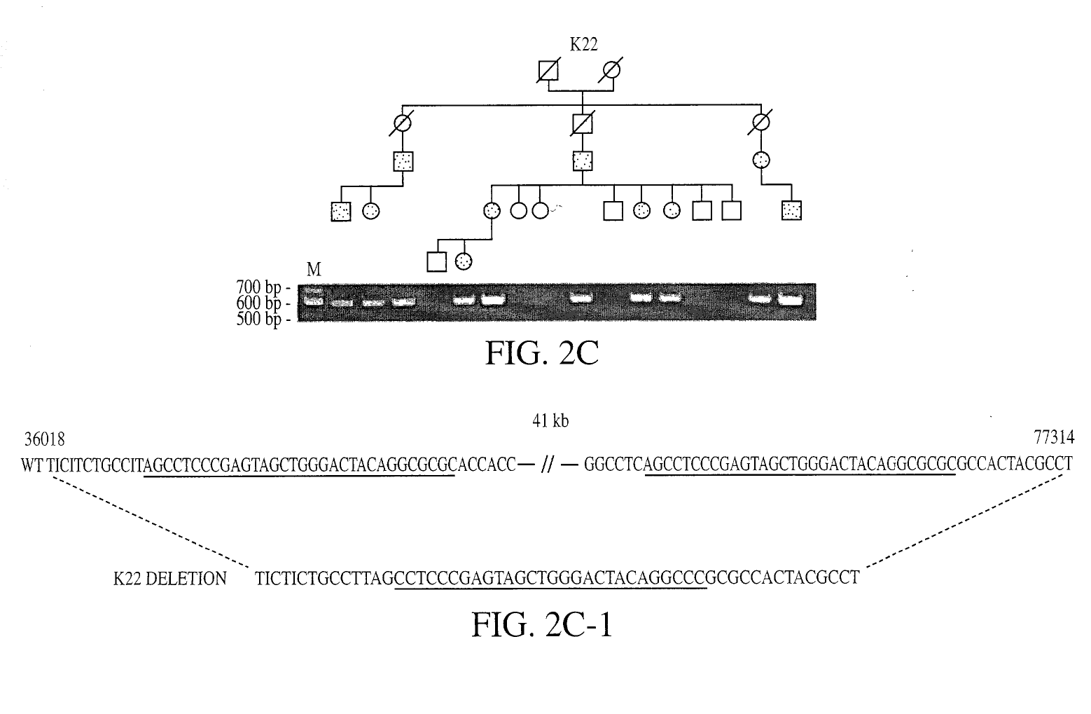

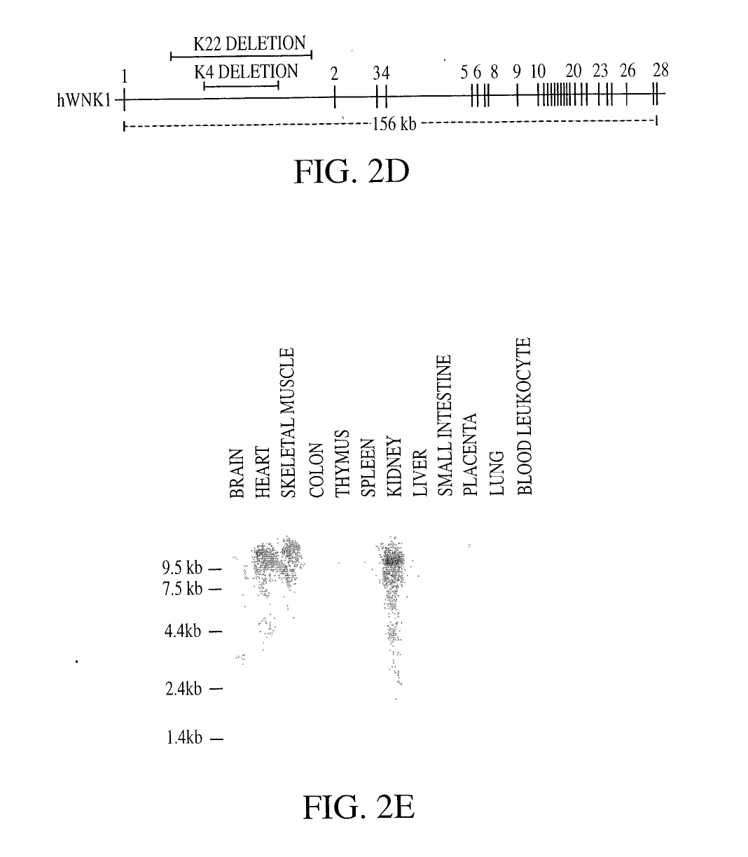

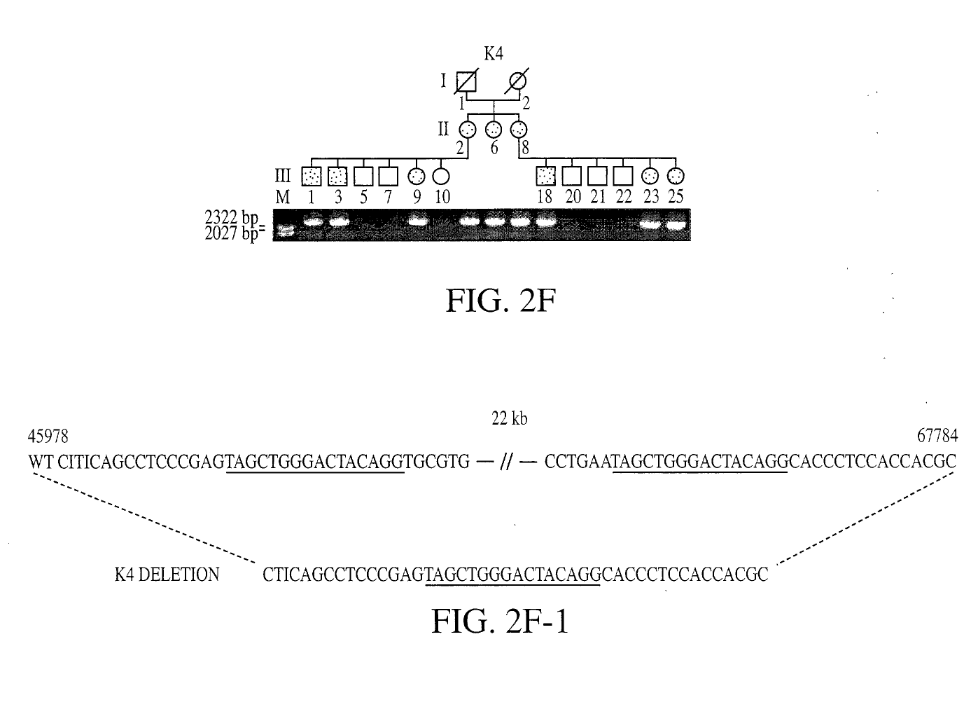

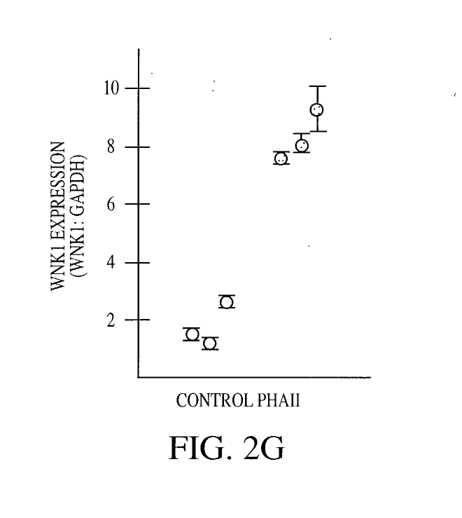

Characterization of the deletion in the K22 kindred: The endpoints of the deletion interval were determined by Southern blotting with genomic DNA from affected and unaffected members of K22. Probes and restriction enzyme cleavage fragments designed from the BAC clone were used to detect a novel 6.9 kb fragment in affected members of the kindred (FIGS. 2A and 2B). This fragment is widely separated in wild-type DNA, confirming the presence of the large deletion identified by linkage analysis.

PCR primers separated by 42 kb in genomic DNA from unaffected members of the kindred and from 160 unrelated controls were used to amplify a genomic DNA template from affected members of the kindred, resulting in a 600 bp fragment. This result indicates that a 41,241 bp deletion in cosegregates with PHA II in the K22 kindred (FIG. 2C). Similar analysis of the K4 kindred indicated that a 21,761 bp deletion occurs within the larger K22 deletion (FIG. 2D). No deletions overlapping with the K22 segment were found in control subjects. The results from both kindreds indicate that a large genomic deletion is responsible for PHA II.

Comparison of PCR product sequences and publicly available genomic databases revealed that the deletion lies in the large, first intron of the human ortholog of rat WNK1 (Xu et al., 2000, J. Biol. Chem., 275:16795). WNK1 is a serine-threonine kinase distinctive because of the substitution of a cysteine for a lysine in a key active site position. The human WNK1 gene is encoded by 28 exons spanning 156 kb in the human genome (FIG. 2D). Rat and human WNK1 are 86% identical at the primary amino acid level.

Expression analysis of hWNK1: Quantitative RT-PCR was used to compare transcript RNA transcript levels from affected members of K4 and from two unaffected members of K4 as well as unrelated controls. Individuals affected with PHA II had hWNK1 transcript levels five times higher than that of unaffected kindred members or unrelated controls, indicating that the intron deletion in hWNK1 alters expression.

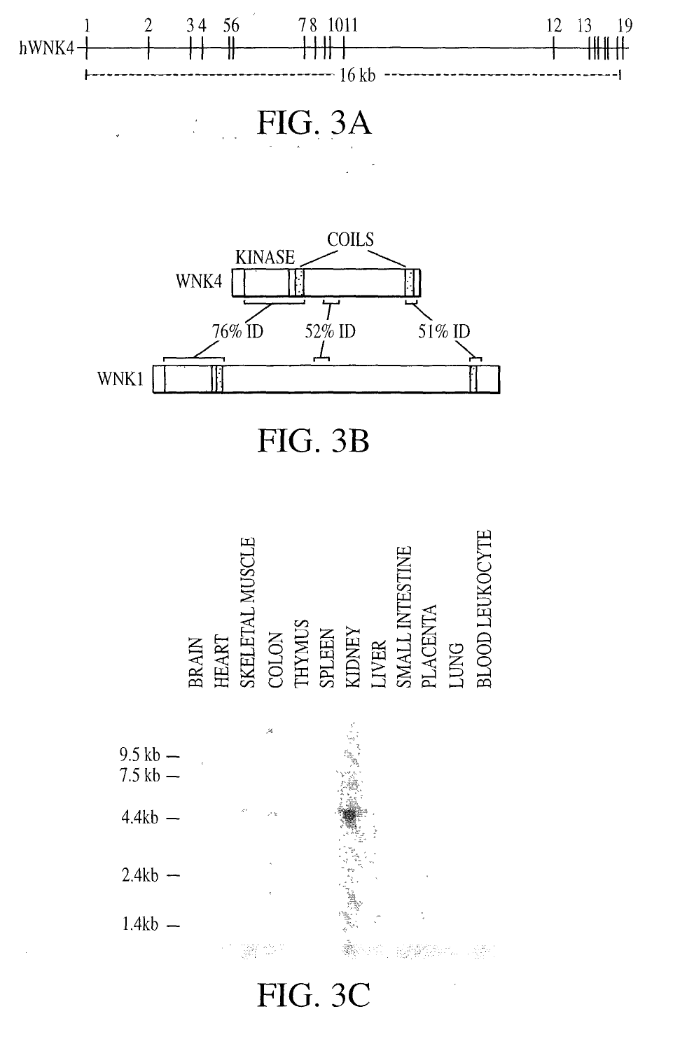

Identification and characterization of hWNK4: Additional PHA II related loci have been mapped to chromosomes 17 and 1 (Mansfield et al., 1997, Nature. Genet. 16:202). A search of publicly available genome databases indicated that a paralog of hWNK1 exists on chromosome 17 between loci D17S250 and D17S579, both of which lie within the minimum genetic interval containing the PHA IIB locus. The chromosome 17 paralog of hWNK1 was identified as hWNK4 (SEQ ID NO:4). hWNK4 is encoded by 19 exons within 16 kb of genomic DNA, and demonstrates 76% identity to hWNK1 across a 370 amino acid segment spanning the kinase and first putative coil domain (FIGS. 3A and 3B). hWNK4 also has the unique cysteine substituted for lysine seen throughout the kinase domain of all WNK serine/threonine kinases.

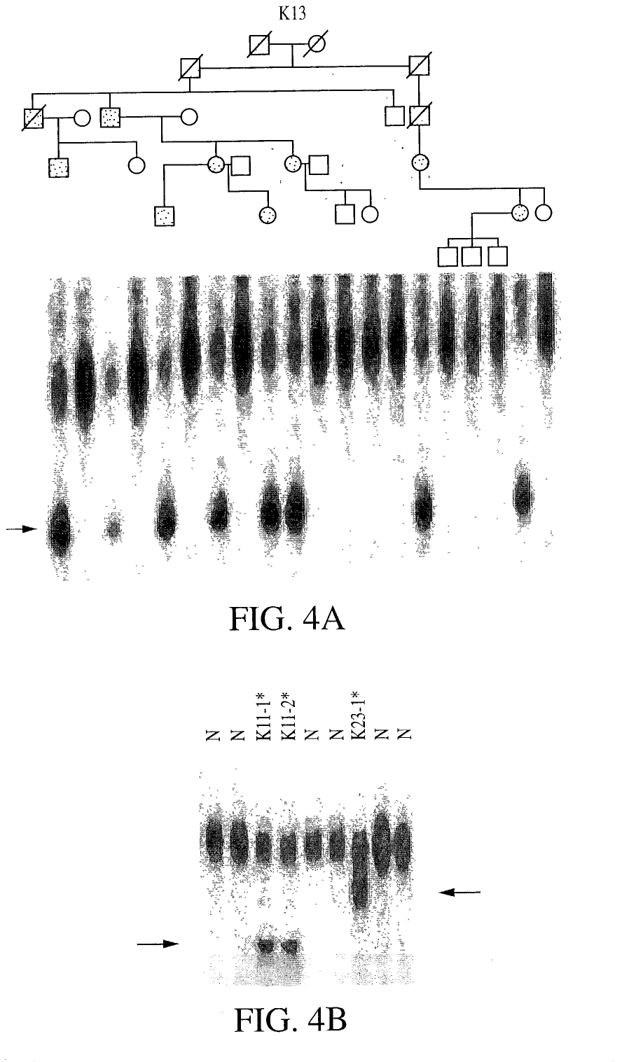

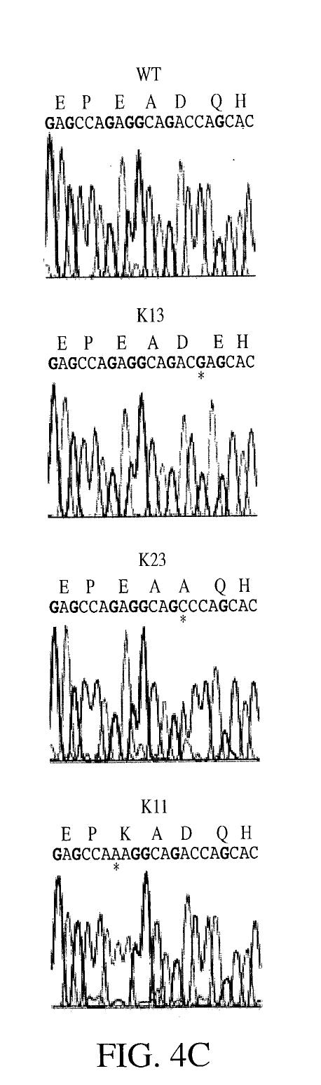

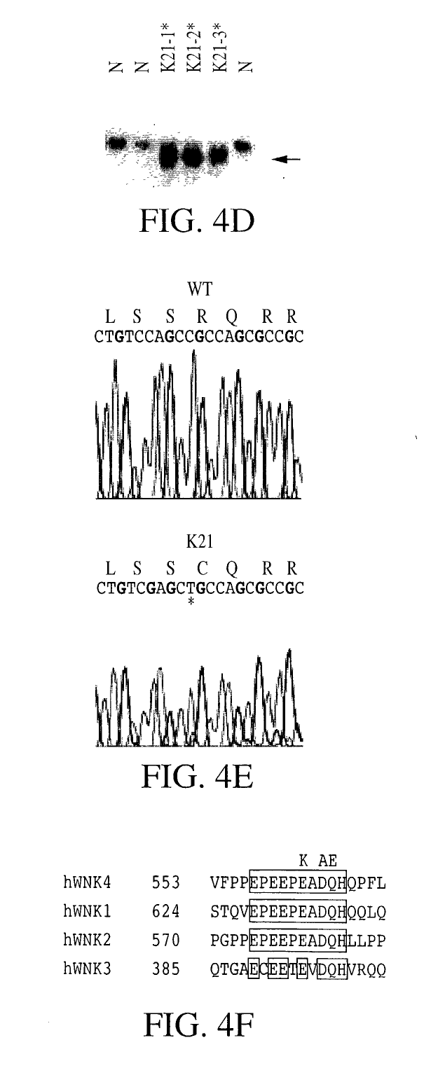

Single-stranded conformational polymorphism assays of hWNK4 in PHA II kindreds identified four missense mutations, all of which co-segregated with the disease (FIG. 4A and 4B). Three of the mutations change the type and charge of the amino acid just distal to the first putative coil domain. Similar mutations are seen in other PHA II kindreds. In K13, all eight affected members, but none of the unaffected members of the kindred inherited a Gln⁵⁶⁵ to Glu mutation. In K23 and K11, Asp⁵⁶⁴ is changed to an Ala and Glu⁵⁶² is changed to a Lys, respectively.

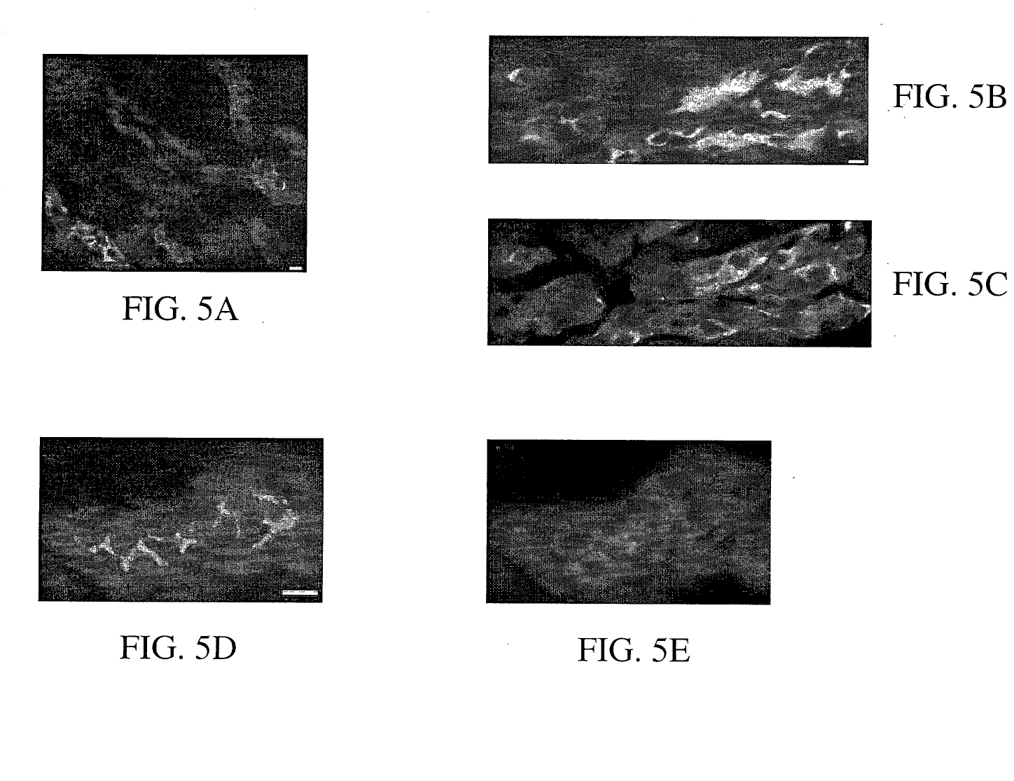

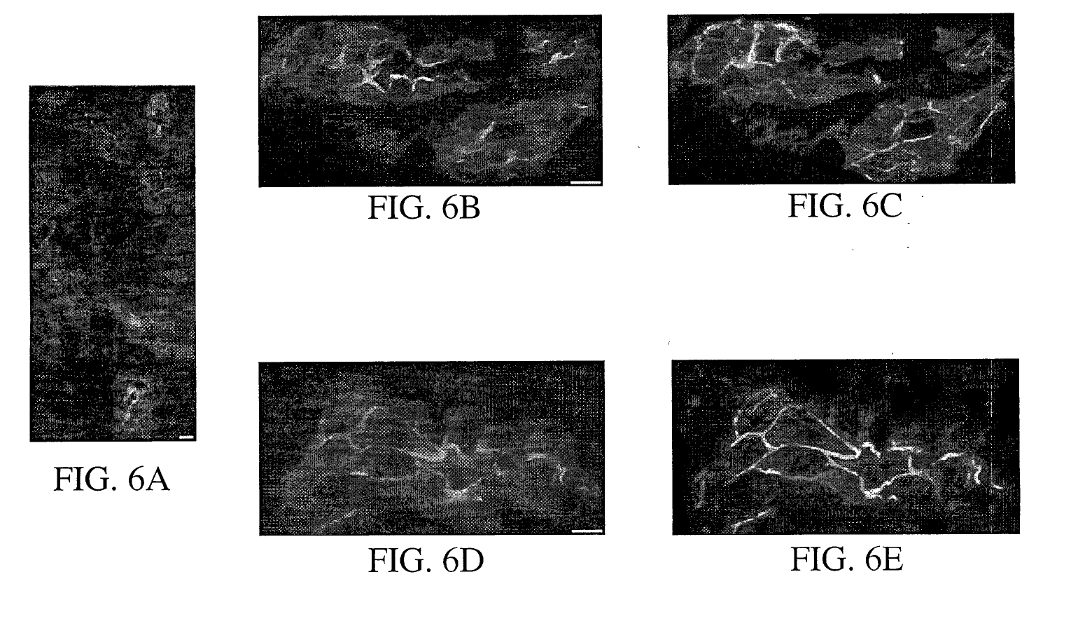

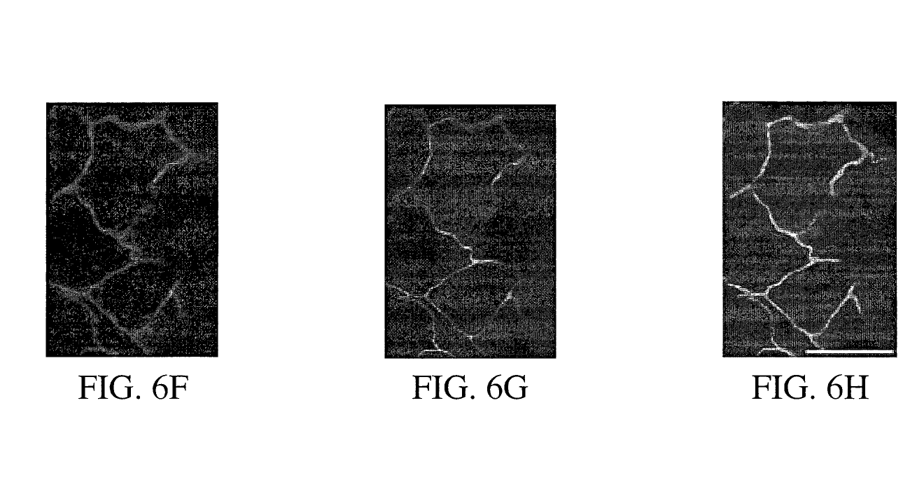

Localization of WNK1 and WNK4: Immunofluorescence of mouse kidney sections employing anti-WNK1 and WNK-4 polyclonal antibodies demonstrated that both proteins localize to the distal convoluted tubule (DCT) and the cortical collecting duct (CCD) (FIGS. 4A and 5A). The DCT and CCD are adjacent segments of the distal nephron that are key in salt, potassium and pH homeostasis. WNK1 is also prominent in the medullary collecting duct, but neither protein was detected elsewhere in the kidney.

The subcellular distribution of each protein was similarly investigated. WNK1 is present throughout the cytoplasm (FIG. 4B), while WNK4 is present exclusively in the intracellular junctions of the DCT and both the cytoplasm and intracellular junctions of the CCD (FIG. 5B). To this end, WNK4 also co-localizes with ZO-1, a well known tight junction protein, indicating that WNK4 is part of the tight junction complex. The tight junction complex is known as an important regulator of paracellular ionic flux (Madara, 1998, Annu. Rev. Physiol., 60:143), and mutations in the protein components of the tight junction complex have been demonstrated to alter ionic flux (Simon et al., 1999, Science, 285:103).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. An isolated nucleic acid encoding a mammalian WNK, wherein said nucleic acid shares greater than 86% identity with at least one of SEQ ID NO:1 and SEQ ID NO:3.

2. An isolated nucleic acid encoding human WNK, said nucleic acid having greater than 86% identity with a nucleic acid selected from the group consisting of SEQ ID NO:1 and SEQ ID NO:3.

3. An isolated nucleic acid encoding a mammalian WNK, wherein said nucleic acid is selected from the group consisting of an isolated nucleic acid consisting of the nucleotide sequence of SEQ ID NO:1, and an isolated nucleic acid consisting of the nucleotide sequence of SEQ ID NO:3.

4. An isolated nucleic acid encoding a mammalian WNK, wherein said nucleic acid encodes human WNK1 and further wherein said nucleic acid shares greater than 86% identity with a nucleic acid having the nucleic acid sequence of SEQ ID NO:1, and further wherein said nucleic acid comprises a deletion of at least a portion of intron 1 of the genomic DNA encoding said human WNK1.

5. The isolated nucleic acid of claim 4, wherein said deletion consists of a deletion from about nucleotide number 36018 to about nucleotide number 77314 relative to the sequence of BAC clone GenBank accession number AC004765.

6. The isolated nucleic acid of claim 1, said nucleic acid further comprising a nucleic acid encoding a tag polypeptide covalently linked thereto.

7. The isolated nucleic acid of claim 1, said nucleic acid further comprising a nucleic acid specifying a promoter/regulatory sequence operably linked thereto.

8. A vector comprising the nucleic acid of claim 1.

9. A recombinant cell comprising the isolated nucleic acid of claim 1.

10. An isolated nucleic acid complementary to an isolated nucleic acid encoding a mammalian WNK, or a fragment thereof, said complementary nucleic acid being in an antisense orientation.

11. The isolated nucleic acid of claim 11, wherein said nucleic acid shares greater than 86% identity with a nucleic acid complementary with a nucleic acid having the sequence of at least one of a human WNK1 (SEQ ID NO:1), and a human WNK4 (SEQ ID NO:3).

12. An isolated mammalian WNK polypeptide.

13. An isolated mammalian WNK, wherein said WNK comprises an amino acid sequence having greater than 86% identity with a polypeptide having the amino acid sequence selected from the group consisting of an amino acid having the sequence of SEQ ID NO:2, and an amino acid having the sequence of SEQ ID NO:4.

14. An isolated human WNK, wherein said WNK is selected from the group consisting of human WNK1 and human WNK4.

15. The isolated human WNK of claim 13, wherein said human WNK is hWNK4 and further wherein said hWNK4 comprises a mutation selected from the group consisting of an amino acid substitution at amino acid residue number 565 from a glycine to a glutamine relative to the sequence of SEQ ID NO:4, an amino acid substitution at amino acid residue number 56 from aspartic acid to alanine relative to the sequence of SEQ ID NO:4, an amino acid substitution at amino acid residue number 562 from a glutamine to lysine relative to the sequence of SEQ ID NO:4, and an amino acid substitution at amino acid residue number 1185 from arginine to cysteine relative to the sequence of SEQ ID NO:4.

16. An antibody that specifically binds with a mammalian WNK, or a fragment thereof.

17. The antibody of claim 16, wherein said mammalian WNK shares greater than about 86% identity with a polypeptide having the amino acid sequence selected from the group consisting of SEQ ID NO:2 and SEQ ID NO:4.

18. The antibody of claim 16, wherein said antibody is selected from the group consisting of a polyclonal antibody, a monoclonal antibody, and a synthetic antibody.

19. A composition comprising the isolated nucleic acid of claim 1 and a pharmaceutically-acceptable carrier.

20. A composition comprising the isolated polypeptide of claim 12 and a pharmaceutically-acceptable carrier.

21. A method of identifying a compound that inhibits expression of human WNK in a cell, said method comprising contacting a cell with a compound and comparing the level of expression of human WNK in said cell contacted with said compound with the level of expression of human WNK in an otherwise identical cell, wherein a lower level of expression of human WNK in said cell contacted with said compound compared with the level of expression of human WNK in said otherwise identical cell not contacted with said compound, is an indication that said compound inhibits expression of human WNK in said cell.

22. The method of claim 21, wherein said human WNK is selected from the group consisting of hWNK1 and hWNK4.

23. A compound identified by the method of claim 21.

24. A method of treating a disease mediated by expression of a human WNK, said method comprising administering to a human patient afflicted with a disease mediated by expression of a human WNK, a human WNK expression-inhibiting amount of a WNK inhibitor, thereby treating a disease mediated by expression of a human WNK.

25. The method of claim 24, wherein said disease is selected from the group consisting of hypertension and pseudohypoaldersteronism type II.

26. The method of claim 25, wherein said disease is pseudohypoaldersteronism type II and further wherein said mammal is a human.

27. The method of claim 24, wherein said WNK inhibitor comprises an isolated nucleic acid complementary to an isolated nucleic acid encoding a human WNK, or a fragment thereof, said complementary nucleic acid being in an antisense orientation.

28. A method of treating hypertension in a mammal, wherein said hypertension is mediated by increased expression of a mammalian WNK1, said method comprising administering to a mammal afflicted with a disease mediated by increased expression of a mammalian WNK1, a WNK1 expression-inhibiting amount of a WNK inhibitor, thereby treating hypertension in said mammal.

29. A method of treating pseudohypoaldersteronism type II in a mammal, wherein said pseudohypoaldersteronism type II is mediated by increased expression of a mammalian WNK1, said method comprising administering to a mammal afflicted with pseudohypoaldersteronism type II a WNK expression-inhibiting amount of a WNK inhibitor, thereby treating pseudohypoaldersteronism type II in said mammal.

30. A method of treating hypertension in a mammal, wherein said hypertension is mediated by expression of a mutant mammalian WNK4, said method comprising administering to a mammal afflicted with a disease mediated by expression of a mutant mammalian WNK4, a WNK expression-inhibiting amount of a WNK inhibitor, thereby treating hypertension in said mammal.

31. A method of treating pseudohypoaldersteronism type II in a mammal, wherein said pseudohypoaldersteronism type II is mediated by expression of a mutant mammalian WNK4, said method comprising administering to a mammal afflicted with pseudohypoaldersteronism type II, a WNK expression-inhibiting amount of a WNK inhibitor, thereby treating pseudohypoaldersteronism type II in said mammal.

32. A method of identifying a human patient afflicted with a disease, disorder or condition associated with altered expression of WNK, said method comprising detecting the level of WNK expression in a human and comparing said level of expression of WNK in said human with the level of expression of WNK in a normal human not afflicted with a disease, disorder or condition associated with altered expression of WNK, thereby detecting a human patient afflicted with a disease, disorder or condition associated with altered expression of WNK.

33. The method of claim 32, wherein said disease, disorder or condition associated with altered expression of WNK is selected from the group consisting of hypertension and pseudohypoaldersteronism type II.

34. A method of detecting a mutation in a WNK allele in a human, said method comprising comparing the nucleic acid sequence encoding WNK of a human suspected of having a mutation in WNK with the nucleic acid sequence encoding WNK obtained from a normal human not having a mutation in WNK, wherein any difference between said nucleic acid sequence of said human suspected of having a mutation in WNK and said nucleic acid sequence encoding WNK of said normal human not having a mutation in WNK detects a mutation in a WNK allele in said human.

35. The method of claim 34, wherein said WNK is human WNK4 and further wherein said mutation is selected from the group consisting of an amino acid substitution at amino acid residue number 565 from a glycine to a glutamine relative to the sequence of SEQ ID NO:4, an amino acid substitution at amino acid residue number 564 from aspartic acid to alanine relative to the sequence of SEQ ID NO:4, an amino acid substitution at amino acid residue number 562 from glutamine to lysine relative to the sequence of SEQ ID NO:4, and an amino acid substitution at amino acid residue number 1185 from arginine to cysteine relative to the sequence of SEQ ID NO:4.

36. A method of detecting a mutation in a WNK allele in a human, said method comprising comparing the genomic nucleic acid sequence encoding WNK of a human suspected of having a mutation in WNK with the genomic nucleic acid sequence encoding WNK obtained from a normal human not having a mutation in WNK, wherein any difference between said genomic nucleic acid sequence of said human suspected of having a mutation in WNK and said genomic nucleic acid sequence encoding WNK of said normal human not having a mutation in WNK detects a mutation in a WNK allele in said human.

37. The method of claim 36, wherein said WNK is hWNK1, and further wherein said mutation comprises deletion of at least a portion of intron 1.

38. The method of claim 37, wherein said deletion consists of a deletion relative to the sequence of BAC clone GenBank accession number AC004765.
